(19) United States
(12) Patent Application Publication
(10) Pub. No.: US 2010/0261682 A1
(43) Pub. Date: Oct. 14, 2010

(54) Method and Means for the Treatment of Cachexia

(75) Inventors: Matti Sirén, Helsingfors (FI); Pontus Gallen-Kallela-Sirén, Helsingfors (FI)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(73) Assignee: Bioneris AB

(21) Appl. No.: 12/676,173

(22) PCT Filed: Sep. 17, 2008
(86) PCT No.: PCT/SE2008/051043

§ 371 Date: Jun. 8, 2010

Related U.S. Application Data
(60) Provisional application No. 60/994,061, filed on Sep. 17, 2007.
(60) Provisional application No. 61/050,778, filed on May 6, 2008.

(30) Foreign Application Priority Data
Sep. 17, 2007 (SE) 0702076-1

(51) Int. Cl. A61K 31/6615; A61P 3/00

(52) U.S. Cl. 514/144

(57) ABSTRACT

The present invention relates to the treatment of cachexia in a mammal by the use of a compound comprising a high density negatively charged domain of vicinally oriented radicals.

### FIELD OF THE INVENTION

The present invention relates to the treatment of cachexia and a corresponding means.

### BACKGROUND

The breakdown of lean body tissue, cachexia, is a serious problem that occurs in a number of acute and chronical clinical conditions. Side effects of various medical treatments can also lead to cachexia. Trauma, surgery, burn injury, injury, prolonged fasting, sepsis, prolonged bed rest, cancer and AIDS are examples of catabolic states that can lead to a significant loss of lean body tissue and skeletal muscle. Protein catabolism (cachexia) leads to the acceleration of protein degradation and an elevation of energy expenditure or hypercatabolism. Further, catabolism is often associated with elevated urinary nitrogen excretion which leads to a negative nitrogen balance.

Although cachexia causes the depletion of both adipose and muscle tissue, muscle atrophy is the most important prognostic factor in determining the survival of patients who suffer from cachexia. The catabolic response in muscles results in muscle tissue wasting and increased fatigue and severely influences the quality of life of the patients. The degree of muscle wasting has also been shown to correlate with a poor response to overall therapy. Specifically, the response to chemotherapy is impaired in patients with cancer cachexia (van Eys, Annu Rev Nutr 435-461, 1985).

The cachexia related catabolic response in skeletal muscle is primarily caused by stimulated protein breakdown and especially by the breakdown of the myofibrillar protein. This increased protein breakdown is accompanied by decreased protein synthesis which contributes to the negative protein balance in muscle tissue.

Intracellular protein breakdown is regulated by several proteolytic pathways including a) lysomal b) Ca-dependent and c) ubiquitin-proteasome dependent mechanisms. Recent studies in rats and mice models suggest that muscle proteolysis is regulated mainly by the ubiquitin-proteasome pathway and is associated with the up-regulation of several genes in this pathway. A similar mechanism which has been analyzed in detail in in vivo and in vitro models has also been identified to be involved in human cachexia syndrome. The ubiquitin proteasome metabolic pathway which has been identified in muscle wasting, is activated in various pathological states such as cancer, sepsis, and burn injury among others. These conditions show an accelerated ubiquitin-mediated proteolysis. The first report of increased expression of genes in the ubiquitin-proteasome proteolytic pathway in the muscle tissue of human cancer cachexia patients was published in 1999 (Williams A., et al. Surgery, 744-750, 1999). The mRNA levels for ubiquitin and the 20 S proteasome subunits were 2 to 4 times higher in muscle from patients with cancer than in muscle from control patients.

Loss of lean body mass in catabolic illness, cachexia, can have a very significant impact on the clinical course and outcome in affected patients. Changes in the ratio of lean body mass to body fat can markedly alter drug distribution and pharmacokinetics and at the same time reduce drug efficacy and increase toxicity and side effects. Lean body wasting can also impair immune function and increase the risk of sepsis. A significant percentage of cancer and AIDS patients suffer from a severe catabolic condition known as cachexia. The mechanism leading to tissue breakdown is still unclear, but it has been postulated that the effect of the catabolism is to increase the inter- and intracellular supply of amino acids. A number of factors appear to be involved in catabolic conditions, such as altered ratios of anabolic/catabolic hormones, reduced sensitivity of tissues to anabolic hormones and endogenous cytokines such as interleukins.

Physiologic and metabolic changes that usually accompany catabolic conditions are for example increased proteolysis, altered carbohydrate metabolism, increased fat oxidation, increased whole body protein turnover, anorexia, impaired immune response, decreased wound healing and altered drug pharmacokinetics. The clinical treatment of lean body wasting in catabolic illness still focuses primarily on the provision of specialized enteral and parenteral nutrition. However, a number of studies have shown that nutritional therapy alone is relatively ineffective at reducing net protein breakdown or stimulating protein synthesis during catabolic illness. Thus, there is a need for additional agents directed at reversing protein losses and restoring the balance of protein metabolism.

Cachexia or Wasting Syndrome is frequently associated with terminal cancer, but also with AIDS, Congestive Heart Failure, Chronic Obstructive Pulmonary Disease, Sepsis, Uremia, Acidosis, Diabetes mellitus and other conditions (Hasselgren P O J Biochem & cell Biol 2156-2168, 2005). Cachexia significantly amplifies the impact of the primary condition and contributes to the morbidity associated with these diseases. Cachexia is common in cancer patients, but not all types of tumours produce cachexia. Independent of the tumour disease, the reduction of lean body mass in a cachectic patient may be life-threatening, in particular due to the impairment of respiratory muscle function.

Cachexia results from the imbalance in protein degradation and protein synthesis. In cachexia patients, protein synthesis is depressed and protein degradation is increased, leading to an imbalance in the protein metabolism in the muscles. Recent research suggests that the mechanisms causing muscle atrophy include the activation of the ATP-ubiquitin-proteasome proteolytic pathways which leads to accelerated protein degradation of muscle protein and inhibition of protein synthesis. Currently there is no therapy which effectively addresses this fundamental metabolic imbalance.

The causes of cachexia have been poorly understood. It is though widely believed that inflammatory cytokines like tumour necrosis factor alpha (TNF-α), interferon gamma (IFN-γ), and interleukin 6 (IL-6) are involved in cachexia. In addition, Proteolysis-Inducing Factor (PIF) has been associated with cachexia (T M Watchorn et al., Proteolysis-inducing factor regulates gene expression via the transcription factors NF-κB and STAT3. FASEB J 2001; 15:562-564). High levels of the appetite-stimulating hormone ghrelin are found in cachectic cancer patients (G M Garcia et al., Active Ghrelin Levels and Active to Total Ghrelin Ratio in Cancer-Induced Cachexia. J Clin Endocrinol Metab 90:5 (2005) 2920-2926).

Various treatments of cachexia are known in the art, such as a treatment based on the suppressive action of Tumour Cytotoxic Factor II of TNF (U.S. Pat. No. 7,138,372 B2); on the induction of an anti-tumour and anti-cachexia immune response (US 2004/0228925 A1); on the administration of certain unsaturated fatty acids, in particular eicosapentaenoic acid (EP 0 464 084 B1), of p-hydroxy-β-methylbutyrate (H J Smith et al., Attenuation of Proteasome-Incuced Proteolysis in Skeletal Muscle by β-hydroxy-β-methylbutyrate in Cancer-Induced Muscle Loss. Cancer Res 65 (2005) 722-283); of megestrol, a synthetic progestin (U.S. Pat. No. 7,101,576 B2), of inhibitors of the renin-angiotensin system (U.S. Pat. No. 7,071,183 B2; Sanders, P M et al., Angiotensin II directly induces muscle protein catabolism through the ubiquitin-proteasome proteolytic pathway and may play a role in cancer cachexia. Brit J Cancer 93 (2005) 425-434); of ghrelin and ghrelin-like compounds (US 2007/0037751 A1); of melanocortin-4 receptor agonists (US 2006/0014676 A1); of hydrazine sulphate (U.S. Pat. No. 5,264,208 A); and others. However, none of the disclosed treatments is fully satisfactory, and furthermore some of them may be accompanied by severe side effects. Therefore, the need for an improved or alternative treatment of cachexia, in particular cancer cachexia, still exists.

Despite the great clinical need for a cachexia treatment, there are no effective pharmaceutical products which effectively treat cachexia. This absence of therapy is significant because despite the existence of many treatment regimes against cancer as such, cachexia continues to be a major factor limiting the successful overall treatment of cancer patients. Cachexia significantly interferes with the effectiveness of the other anti-cancer treatments. Cancer cachexia is not simply a local effect of the tumour. Alterations in protein, fat, and carbohydrate metabolism occur commonly. For example, abnormalities in carbohydrate metabolism include increased rates of total glucose turnover, increased hepatic gluconeogenesis, glucose intolerance and elevated glucose levels. Increased lipolysis, increased free fatty acid and glycerol turnover, hyperlipidemia, and reduced lipoprotein lipase activity are frequently noted. The weight loss associated with cancer cachexia is caused not only by a reduction in body fat stores but also by a reduction in total body protein mass, with extensive skeletal muscle wasting. Increased protein turnover and poorly regulated amino acid oxidation is also important components.

For the majority of patients whose cancer has spread beyond the organ of origin, neither surgery, radiotherapy nor chemotherapy is able to offer a cure. With the recognition of the morbidity and mortality associated with cachexia, the past 15 years have seen attempts to use nutritional support to try to reverse the nutritional deficit associated with progressive cancer growth. These studies have, however, not been successful. Conventional nutritional support does not readily reverse the nutritional deficits associated with progressive tumour growth and nutritional support has failed to reduce overall morbidity and mortality (Fearon et al., 1991).

Traditionally, anti-cachexia treatments have: 1) targeted either the primary tumour tissue with the objective to inhibit or slow the tumour growth, or 2) have sought to inhibit the metabolic effect produced by the primary tissue which causes wasting in secondary tissues either by inhibiting the release of cachexia inducing factors by the tumour or by inhibiting the effect of these factors on the target secondary tissue. Historically, a serious limitation of cachexia research has been the lack of a good animal model which would have allowed the examination of the molecular pathways of cachexia and which could also have been used to test prospective anti-cachexic compounds. An animal model is the most effective and reliable way to study the impact of the tumour on wasting of peripheral tissue such as muscle tissue. Cachexia is an affliction of the tumour bearing host and therefore the study of cachexia is possible only in in vivo models and necessitates the study of the entire, living tumour bearing animal.

In the late 1980's, an animal model was been developed which consistently induces cachexia in experimental animals. The “MAC16” animal model, which uses NMRI mice and an adenocarcinoma cell line, consistently induces rapid tumour growth and cachexia. The cachexia has been shown to result from the depression of protein synthesis (60%) and increased protein degradation (240%) (Tisdale et al., 1993 Br J Cancer). The metabolic imbalance in the skeletal muscle resulting from the depression of protein synthesis and increased protein degradation releases increased amounts of amino acids and inorganic elements into the blood stream. It is possible that the primary tumour can use these nutrients for growth and proliferation. Paradoxically, the peripheral muscles may serve as a nutrient reservoir for the primary tumour. These nutrients are made available to the primary tumour through the increased degradation of the muscle tissue.

Today, it is possible to accurately measure the protein synthesis and protein degradation both in in vitro and in vivo conditions. The effective testing, both in vitro and in vivo, of anti-cachexia agents has developed significantly over the past years. These developments in research techniques have enabled the screening of potential anti-cachexia agents.

The MAC16 model has been used to search for tumour specific substances which are produced by the tumour tissue and which influence the wasting metabolism of host cells. One such product was named proteolysis inducing factor (PIF) which was discovered in 1996 (Tisdale et al., Nature. 1996 Feb.). PIF was named after its metabolic effect and its molecular pathway has been largely elucidated.

Cachexic cancer patients have measurable quantities of PIF in their urine, whereas healthy controls don't. PIF is not found in the urine of weight-stable cancer patients or in weight-losing controls with benign tumours. The primary tumour tissue secretes PIF into the blood stream, and PIF causes wasting of the skeletal muscles. PIF has been shown to cause both the depression of protein synthesis in skeletal muscle and the increase in protein degradation (Tisdale et al., Skeletal muscle atrophy, a link between depression of protein synthesis and increase in degradation. Journal of Biological Chemistry 2007 Mar. 9; (10):7087-7097). Only cachexia-inducing tumours are capable of elaborating fully glycosylated PIF (Tisdale M., Tumour-host interactions. J of Cell Biochemistry, 2004 Nov. 15; 93(5):871-7). PIF promoted the enhanced protein degradation in the soleus muscle of mice bearing the MAC16 tumour, confirming that PIF is responsible for the loss of skeletal muscle in cachexic mice. When PIF was given to non-tumour bearing mice, it induced a significant loss of protein from muscle tissue, suggesting that PIF may be responsible for affecting changes leading to cachexia (Bhogal a et al., Changes in nucleic acid and protein levels in atrophying skeletal muscle in cancer cachexia. Anticancer Res. 2006 November-December; 26:4149-54). Furthermore, in vitro models show that besides activating the proteasome, PIF induces apoptosis in C(2)C(12) myotubes. Both of these processes contribute to the loss of skeletal muscle in cancer cachexia (Smith H., Induction of apoptosis by a cachexic-factor in murine myotubes and inhibition by eicosapentaenoic acid. Apoptosis, 2003 March; 8(2):161-9). All these results suggest that PIF is a principal factor mediating changes in skeletal muscle homeostasis leading to cancer cachexia. The proteolytic effects of PIF in muscle tissue have been extensively studied in vitro, and the pathways related to the increased protein degradation and decreased protein synthesis have been detailed. PIF provides an important link between the tumour tissue and skeletal muscle and helps explain how the tumour tissue causes wasting of the peripheral skeletal muscles.

Another key substance identified as a causative agent in cancer cachexia is Angiotensin II (Ang II). This is surprising as Ang II has traditionally been associated with cardiovascular organs such as the heart and vessel walls. Ang II is known to regulate blood pressure and electrolyte and fluid balance in organisms. Ang II is the main bioactive component of the rennin Angiotensin system and is formed from the precursor molecules angiotensinogen and Ang I. Angiotensin converting enzyme (ACE) converts Ang I into Ang II. Recent studies have shown that the chymase enzyme produces the same result as ACE and can convert Ang I into Ang II. Mammalian chymase was originally identified in mast cells (Sayama et al, Human chymotrypsin-like proteinase chymase sub-cellular localization to mast cell granules and interaction with heparin and their glycoaminoglycans. J Biol Chem 263, 1987, 6808) and chymase is known to be the main protein in mast cells granules (Katuma et a, Eur j Biochem, 52, 1975, 37). Mast cells are widely distributed in tissue, especially in the connective tissue of vertebrates.

Ang II, like PIF, induces wasting of skeletal muscle. Angiotensin II has been directly linked to cachexia and shown to significantly inhibit protein synthesis in murine myotubes (Tisdale M et al., Angiotensin II directly inhibits protein synthesis in murine myotubes. Cancer Letters, 2006 Jan. 18; 231(2):290-294). Angiotensin II infusion in the rat produces cachexia, and Ang II has been shown to stimulate protein degradation in myotubes through induction of the ubiquitin-proteasome pathway suggesting that Ang II can cause muscle atrophy and cachexia. (Brink M., Angiotensin II induces skeletal muscle wasting through enhanced protein degradation and down-regulates autocrine insulin-like growth factor I. Endocrinology. 2001 April; 142:1489-96 and Sanders P M., et al., Angiotensin II directly induces muscle protein catabolism through the ubiquitin-proteasome proteolytic pathway and may play a role in cancer cachexia, Br J Cancer. 2005 Aug. 22; 93:425-34). Ang II has also been shown to have the ability to induce muscle atrophy through the inhibition of protein synthesis (Russell S T. et al., Angiotensin II directly inhibits protein synthesis in murine myotubes, Cancer Lett. 2006 Jan. 18; 231:290-4).

Both Ang II and PIF have been found in human patients suffering from cachexia. Both molecules have also been shown to cause cachexia in experimental animals by both promoting protein degradation and inhibiting protein synthesis. These experimental results suggest that Ang II and PIF have a causative relationship in the development of cachexia. PIF and Angiotensin II have identical molecular structures in humans and experimental animals. The PIF found in the cachexic mice is a 24 kD sulfated glycoprotein which is exactly the same molecule as that found in the urine of cachexic cancer patients. Similarly Angiotensin II, which is an octapeptide, has an identical composition in humans and experimental animals.

The central role PIF and Ang II play in cachexia suggests that the inhibition of these two molecules could have a significant positive impact in the treatment and prevention of cachexia.

The atrophy of skeletal muscle can be the result of either the depression of protein synthesis, the increase in protein degradation or a combination of these two phenomena (Smith, British Journal of Cancer, 680, 1993). PIF and AngII have also been associated with the reduction in protein synthesis and the induction of protein degradation. These factors have been shown to bring about the depression in protein synthesis in murine myotubes together with an increased phosphorylation of eukaryotic initiation factor 2 (eIF2α). Phosphorylation of eIF2α by PIF and Ang II seems to occur through activation of PKR, since a PKR inhibitor attenuated the inhibitory effect of both agents on protein synthesis. (Eley, Journal of Biological Chemistry, 7087-7097, 2007).

There are several factors behind the increased protein degradation observed in cachexic animals. Cachexia brings about an increased expression of key elements in the ubiquitin-proteasome pathway. These elements include the 20S proteasome subunits, which has been suggested to be responsible for selective loss of the myofibrillar protein myosin.

Protein degradation has been associated with increased proteasome ‘chymotrypsin-like’ enzyme activity, as well as increased expression of both mRNA and protein for 20S proteasome subunits and the ubiquitin-conjugating enzyme (E2(14k)) (Smith, Biochem Biophys Res Commun. 83-8, 2005). Other factors which have been shown to be play a role in the cachexic pathway are mTOR, the initiation factor 4E-binding protein (4E-BP1), the eukaryotic initiation factor 2 (eIF4E) and eIF4G (Eley, Am J Physiol Endocrinol Metab. E923-31, 2007).

Treatment of C(2)C(12) differentiated, postmitotic multinucleated skeletal myotubes with a tumour-derived proteolysis-inducing factor (PIF) at concentrations between 1 and 10 nM was shown to stimulate the activity of the apoptotic initiator caspases-8 and -9 and the apoptotic effector caspases-2, -3 and -6. Some of the increase in caspase activity has been postulated to be related to the increased proteasome proteolytic activity, since a caspase-3 inhibitor completely attenuated the PIF-induced increase in ‘chymotrypsin-like’ enzyme activity, the predominant proteolytic activity of the proteasome. (Smith, Apoptosis. 161-9 2003)

There remains an unresolved question about the interrelationship between poor nutritional status and a propensity to infection. One of the metabolic hallmarks of sepsis is the catabolic response in skeletal muscle characterized by increased protein breakdown. This catabolic response results in release of amino acids from muscle tissue providing the liver with substrates for acute phase protein synthesis and gluconeogenesis. In severe and protracted sepsis continued muscle protein breakdown results in muscle wasting and fatigue, which may lead to impaired recovery. There is therefore of great clinical significance to understand the mechanisms regulating muscle proteolysis during sepsis for the development of future therapeutic modalities to inhibit the catabolic response in patients with sepsis.

Sepsis is one of the oldest medical terms used to define the serious high frequency morbidity and mortality based inflammatory attack of pathogen microbes occurring after injury which affect critical care and infectious units in hospital as well as in more primitive field ambulatory situations. The most recent up-to-date therapy guidelines for the management of severe sepsis has been published by Philip Dillinger in 2004 (Dellinger et al., Crit Care Med 858-873, 2004). It remains a leading cause of death in many intensive surgical care units. Sepsis is used to denote severe infection and microbiological pathogen infections, but the often fatal end-complications are a metabolic and molecular enigma which do not have an effective therapeutic solution. Today all treatment for sepsis is based on antibiotic therapy, especially intravenous antibiotic therapy against pathogen microbes, fluid therapy, cardiac and circulatory therapy (to restore adequate blood pressure and to increase cardiac output) steroid application, blood product administration and mechanical ventilation.

To our best knowledge, there is today no useable therapeutic agent against cachexia syndrome in the skeletal muscle, such as the diaphragm.

One of the most important metabolic hallmarks this enigma is however the catabolic response in skeletal muscle characterized by increased protein breakdown, in particular myofibrillar protein breakdown of skeletal muscle. This catabolic response results in the release of amino acids from muscle tissue, providing the liver with substrates for acute phase protein synthesis and glucogenesis. The continued muscle protein breakdown results in muscle wasting and fatigue, which may impair recovery and led to increased risk of thromboembolic and pulmonary complications.

A better understanding of the basic mechanisms regulating muscle proteolysis of skeletal muscle is therefore of great clinical significance and is critical for the future development of therapeutic modalities that can inhibit the catabolic response in patients with postoperative pneumonia or atelectasia.

The respiratory muscles are the only skeletal muscles vital for life and the effective impact against protein depletion on respiratory muscle function and locally specific cachexia-phenomenon is a therapeutic approach of this invention. It has been recently confirmed that patients with postoperative complications such as pneumonia or atelectasia, suffer from significant loss of body protein after surgery. The majority of this protein originates from skeletal muscle as evidenced by the net release of amino acids form muscle tissue and urinary exertion of 3-methylhistidine, a marker of myofibrillar protein breakdown. Recent research suggest that muscle protein breakdown during sepsis is caused by the up-regulation of the ubiquitin-proteasome pathway and is associated with the increased expression of the ubiquitin gene.

Studies of septic patients and experimental animals suggest that myofibrillar proteins actin and myosin are particularly sensitive to the effects of sepsis. An understanding of the regulation of these muscle proteins and their breakdown during sepsis and the mechanism involved is very important from a clinical standpoint and is essential for the development of new therapeutic modalities to prevent the loss of muscle tissue (Hasselgren P O, World J Surg, 203-208, 1998).

Knowledge of the central role of the ubiquitin-proteasome pathway in sepsis induced muscle proteolysis has made it possible to design animal models which identify the mechanism of muscle degradation more specifically.

Burn injury is also associated with a negative nitrogen balance and whole body protein loss, mainly reflecting a catabolic response in skeletal muscle. Fang et al have reported that “although previous studies suggest that burn-induced muscle cachexia reflects both inhibition of protein synthesis and increased protein breakdown, the stimulation of protein degradation, in particular myofibrillar protein degradation, is the most important component of muscle catabolism in this condition” (Fang C H, Clin Sci 181-187, 2000).

These and other results suggest that the central pathway in cachexia is the ubiquitin proteasome pathway. As Aaron Ciechanover noted: “The discovery of the ubiquitin pathway and its many substrates and functions has revolutionarized our concept of intracellular protein breakdown” (Ciechanover A, Embo J 7151-7160, 1998).

### OBJECTS OF THE INVENTION

An object of the present invention is to provide an alternative method of preventing, alleviating and/or treating cachexia in particular one that is superior, at least in some respect, to treatments known in the art.

Another object of the present invention is to provide a corresponding means.

Another object of the present invention is to provide a nutritional composition for the prevention or treatment of catabolic conditions, such as cachexia.

Further objects of the invention will become evident from the study of the following summary of the invention, the description of preferred embodiments thereof, and the appended claims.

### SUMMARY OF THE INVENTION

The present invention relates to the use of a compound comprising a high density, negatively charged domain of vicinally oriented radicals for the preparation of a medicament for preventing, alleviating and/or treating cachexia in a mammal. Preferably the negatively charged domain comprises three or more vicinal phosphorus-containing radicals.

In another embodiment the invention relates to a method of treatment of cachexia in a mammal, comprising the administration of a pharmacologically effective amount of a compound, said compound comprising a high density, negatively charged domain of vicinally oriented radicals.

Further preferred embodiments of the present invention are disclosed in the following description and the appended claims.

### DESCRIPTION OF THE INVENTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

In particular, the invention relates to the treatment of catabolic wasting or cachexia, defined as severe catabolic conditions leading to involuntary weight loss. Catabolic wasting, or cachexia, relates to a syndrome characterized by, but not limited to, one or several of the following conditions: involuntary, progressive loss of both fat and skeletal muscle, refractoriness of weight loss to increased nutritional input, elevated resting energy expenditure (REE), decreased protein synthesis, increased protein degradation, altered carbohydrate metabolism, hyper-catabolism of muscle via the ATP-ubiquitin-dependent proteasome pathway of proteolysis, and of adipose tissue via lipolysis. Cachexia occurs in approximately 50% of all cancer patients, either as a direct result of the disease or as a consequence of the treatment (i.e. radiotherapy and/or chemotherapy). The syndrome is also found in patients having e.g., but not limited to, immunodeficiency disorders such as AIDS, cardiac diseases, infectious diseases, patients suffering from bacterial and parasitic diseases, rheumatoid arthritis, chronic diseases of the bowel, liver, kidneys, lungs (e.g. chronic obstructive pulmonary disease) and heart (e.g. chronic heart failure), shock, burn, sepsis, endotoxinimia, organ inflammation, surgery, diabetes, collagen diseases, and trauma. Cachexia can also manifest as a condition in aging and can also be present without an underlying disease. The cachexia syndrome diminishes the patient's functional ability and quality of life, worsens the possible underlying condition and reduces tolerance to medications. The degree of cachexia is inversely correlated with the survival time of patients and it always implies a poor prognosis.

In the context of the present invention the terms “cachexia”, cachectic condition” and cachectic disorder” are used interchangeable.

In the context of the present invention the term “high density” relates to a domain where there is at least two negative charges being distributed among at least two radicals which are connected with covalent bonds to the carbon skeleton.

In the context of the present invention the term “vicinally oriented” relates to radicals being connected to the carbon skeleton to carbon atoms adjacent to each other.

In the context of the present invention the term “radical” relates to a chemical group connected with covalent bonds to the carbon skeleton.

According to the present invention it has surprisingly been possible to use a pharmacologically effective amount of a compound comprising a high density, negatively charged domain of vicinally oriented radicals for the preparation of a medicament for preventing, alleviating and/or treating cachexia in mammals including man. Preferably the domain is at least doubly negatively charged, the two or more charges being distributed between at least two of the radicals. According to a preferred embodiment of the invention the negatively charged domain is capable of complexing divalent cations, such as cadmium, calcium, copper and, in particular, zinc.

The present invention also relates to the use of a pharmacologically effective amount of a compound comprising a high density, negatively charged domain of vicinally oriented radicals for preventing, alleviating and/or treating cachexia in mammals including man.

According to the present invention it is additionally disclosed a method of treatment of cachexia, in a patient in need of such treatment wherein a pharmacologically effective amount of a compound comprising a high density, negatively charged domain of vicinally oriented radicals is administered. The present invention also relates to the use of a pharmacologically effective amount of a compound comprising a high density, negatively charged domain of vicinally oriented radicals for preventing, alleviating and/or treating weight loss associated with cachexia in mammals including man. It is additionally disclosed a method of treatment of weight loss associated with cachexia in a patient in need of such treatment wherein a pharmacologically effective amount of a compound comprising a high density, negatively charged domain of vicinally oriented radicals is administered. In particular cachexia found in patients with cancer as well as cachexia found in conditions like, but not limited to, AIDS, cardiac diseases, infectious diseases, patients suffering from bacterial and parasitic diseases, rheumatoid arthritis, chronic diseases of the bowel, liver, kidneys, lungs and heart, shock, burn, sepsis, endotoxinimia, organ inflammation, surgery, diabetes, collagen diseases, and trauma.

In the context of the present invention is has also surprisingly been found that a compound comprising a high density, negatively charged domain of vicinally oriented radicals can be used for decreasing PIF and AngII induced chymotrypsin-like enzyme activity and for preventing, alleviating and/or treating conditions associated with such enzyme activity.

According to the present invention it is further disclosed a method of inhibiting protein degradation and stimulating protein synthesis in a patient in need of such treatment wherein a pharmacologically effective amount of a compound comprising a high density, negatively charged domain of vicinally oriented radicals is administered.

The present invention will now be further described by the detailed disclosure of the compound that can be used in the embodiments of the invention.

In a preferred embodiment of the invention the negatively charged domain of the compound to be used/administered according to the invention comprises three or more vicinal phosphorus-containing radicals.

According to a preferred embodiment of the invention a phosphorus-containing radical is one of the general formula I

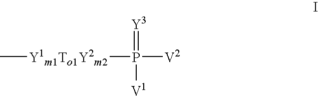

or the general formula II

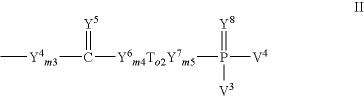

wherein
V¹ to V⁴ are Y⁹ₘ₆Tₒ₃U
Tₒ₁ to Tₒ₃ are (CH₂)ₙ, CH═CH, or CH₂CH═CHCH₂
o1 to o3 are 0 to 1
n is 0 to 4
U is R¹Y¹⁰ₘ₇, CY¹¹Y¹²R², SY¹³Y¹⁴Y¹⁵R³, PY¹⁶Y¹⁷Y¹⁸R⁴R⁵, Y¹⁹PY²⁰Y²¹Y²²R⁶R⁷, CH₂NO₂, NHSO₂R⁸ or NHCY²³Y²⁴R⁹
m1 to m7 are 0 to 1
Y¹ to Y²⁴ are NHR¹², NOR¹¹, O or S
and where R¹ to R¹¹ are
i) hydrogen;
ii) a straight or branched saturated or unsaturated alkyl residue of 1-22 carbon atoms;
iii) a saturated or unsaturated aromatic or non-aromatic homo- or heterocyclic residue of 3-22 carbon atoms and 0-5 hetero atoms selected from nitrogen, oxygen and sulfur;
iv) a straight or branched saturated or unsaturated alkyl residue of 1-22 carbon atoms comprising a saturated or unsaturated aromatic or non-aromatic homo- or heterocyclic substituent of 3-22 carbon atoms and 0-5 hetero atoms selected from nitrogen, oxygen and sulfur;
v) an aromatic or non-aromatic homo- or heterocyclic residue of 3-22 carbon atoms and 0-5 heteroatoms selected from nitrogen, oxygen and sulfur, comprising a straight or branched saturated or unsaturated alkyl substituent of 1-22 carbon atoms.

It is preferred for one or several of the one or more residues and/or substituents of R¹ to R¹¹, groups ii)-v), to be substituted with from 1 to 6 of hydroxy, alkoxy, aryloxy, acyloxy, carboxy, alkoxycarbonyl, alkoxycarbonyloxy, aryloxycarbonyl, aryloxycarbonyloxy, carbamoyl, fluoro, chloro, bromo, azido, cyano, oxo, oxa, amino, imino, alkylamino, arylamino, acylamino, arylazo, nitro, alkylthio, alkylsulfonyl.

It is preferred for one or several of the one or more straight or branched saturated or unsaturated alkyl residues in R¹ to R¹¹, groups ii), iv), v), to be methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methylheptyl, 2-methyloctyl, 2-methylnonyl, 2-methyldecyl, 2-methyleicosyl, 2-ethylbutyl, 2-ethylpentyl, 2-ethylhexyl, 2-ethylheptyl, 2-ethyloctyl, 2-ethylnonyl, 2-ethyldecyl, 2-ethyleicosyl, tert-butyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl, doeicosynyl.

It is preferred for a saturated or unsaturated aromatic or non-aromatic homo- or heterocyclic residue or substituent of R¹ to R¹¹, groups iii)-v), to be selected from cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl, carbohydrate.

According to a first particularly preferred embodiment of the invention a phosphorus-containing radical is one of the general formula III

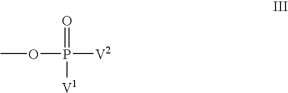

wherein V¹ and V² are, independent of each other, selected from OH, (CH₂)ₚOH, COOH, CONH₂, CONOH, (CH₂)ₚCOOH, (CH₂)ₚCONH₂, (CH₂)ₚCONOH, (CH₂)ₚSO₃H, (CH₂)ₚSO₃, NH₂, (CH₂)ₚNO₂, (CH₂)ₚPO₃H₂, O(CH₂)ₚOH, O(CH₂)ₚCOOH, O(CH₂)ₚCONH₂, O(CH₂)ₚCONOH, (CH₂)ₚSO₃H, O(CH₂)ₚSO₃NH₂, O(CH₂)ₚNO₂, O(CH₂)ₚPO₃H₂, CF₂COOH and p is 1 to 4. In this embodiment of the invention the phosphorus-containing radical is a phosphonate, phosphinate or phosphate including a derivative thereof.

According to this embodiment the domain of high density negatively charged vicinally oriented radicals is linked to a cyclic moiety. The cyclic moiety comprises or consists of a saturated or unsaturated aromatic or non-aromatic homo- or heterocyclic ring. When the moiety comprises a heterocyclic ring the heteroatom(s) thereof are selected from oxygen, nitrogen, sulfur and selenium.

Preferably the cyclic moiety comprises from 4 to 24 atoms, more preferred from 5 to 18 atoms, most preferred 6 atoms. The cyclic moiety is preferably selected from cyclopentane, cyclohexane, cycloheptane, cyclooctane, inositol, monosaccharide, disaccharide, trisaccharide, tetrasaccharide, arabinitol, piperidine, tetra-hydrothiopyran, 5-oxotetrahydrothiopyran, 5,5-dioxotetrahydro-thiopyran, tetrahydroselenopyran, tetrahydrofuran, pyrrolidine, tetrahydrothiophene, 5-oxotetrahydrothiophene, 5,5-dioxotetrahydrothiophene, tetrahydroselenophene, benzene, cumene, mesitylene, naphthalene and phenanthrene. Most preferably the cyclic moiety is selected from the group consisting of inositol, monosacharide, disaccharide, trisaccharide, and tetrasaccharide.

Preferred compounds of the invention when the cyclic moiety is a phosphate, a phosphonate or a phosphinate of cyclohexane are in particular 1,2,3-β-cyclohexane-1,2,3-trioltrisphosphate.

When the cyclic moiety is inositol, which is particularly preferred, it is preferably selected from allo-inositol, cis-inositol, epi-inositol, D/L-chiro-inositol, scylloinositol, myoinositol, mucoinositol and neoinositol.

The inositol is preferably a phosphate, a phosphonate, a phosphinate or derivative thereof. Preferably the number of phosphate, phosphonate or phosphinate radicals per inositol moiety is three or more.

Preferred inositols according to this embodiment are selected from the group consisting of inositol-trisphosphate, inositol-tris(carboxymethyl-phosphate), inositol-tris(carbomethylphosphonate), inositol-tris(hydroxymethylphosphonate), tri-O-methyl-inositol-trisphosphate, tri-O-hexyl-inositol-trisphosphate, tri-O-butyl-inositol-trisphosphate, tri-O-pentyl-inositol-trisphosphate, tri-O-isobutyl-inositol-trisphosphate, tri-O-propyl-inositol-trisphosphate, tri-O-(6-hydroxy-4-oxa)hexyl-inositol-trisphosphate, tri-O-3-(ethylsulfonyl)propyl-inositol-trisphosphate, tri-O-3-hydroxypropyl-inositol-trisphosphate, tri-O-(6-hydroxy)-hexyl-inositol-trisphosphate, tri-O-phenylcarbamoyl-inositol-trisphosphate, tri-O-propyl-inositol-tris(carboxymethylphosphate), tri-O-butyl-inositol-tris(carboxymethylphosphate), tri-O-isobutyl-inositol-tris(carboxymethyl-phosphate), tri-O-pentyl-inositol-tris(carboxymethylphosphate), tri-O-hexyl-inositol-tris(carboxymethylphosphate), tri-O-propyl-inositol-tris(carboxymethylphosphonate), tri-O-butyl-inositol-tris(carboxymethyl-phosphonate), tri-O-isobutyl-inositol-tris(carboxymethylphosphonate), tri-O-pentyl-inositol-tris(carboxymethylphosphonate), tri-O-hexyl-inositol-tris(carboxymethylphosphonate), tri-O-propyl-inositol-tris(hydroxymethyl-phosphonate), tri-O-butyl-inositol-tris(hydroxymethylphosphonate), tri-O-isobutyl-inositol-tris(hydroxymethylphosphonate), tri-O-pentyl-inositol-tris(hydroxymethylphosphonate), and tri-O-hexyl-myo-inositol-tris(hydroxymethyl-phosphonate).

If the inositol is a myo-inositol, preferred compounds are selected from the group consisting of D-myo-inositol-1,2,6-trisphosphate, D-myo-inositol-1,2,6-tris(carboxymethyl-phosphate), D-myo-inositol-1,2,6-tris(carbomethylphosphonate), D-myo-inositol-1,2,6-tris(hydroxymethylphosphonate), D-3,4,5-tri-O-methyl-myo-inositol-1,2,6-trisphosphate, D-3,4,5-tri-O-hexyl-myo-inositol-1,2,6-trisphosphate, D-3,4,5-tri-O-butyl-myo-inositol-1,2,6-trisphosphate, D-3,4,5-tri-O-pentyl-myo-inositol-1,2,6-trisphosphate, D-3,4,5-tri-O-isobutyl-myo-inositol-1,2,6-trisphosphate, D-3,4,5-tri-O-propyl-myo-inositol-1,2,6-trisphosphate, D-3,4,5-tri-O-(6-hydroxy-4-oxa)hexyl-myo-inositol-1,2,6-trisphosphate, D-3,4,5-tri-O-3-(ethylsulfonyl)propyl-myo-inositol-1,2,6-trisphosphate, D-3,4,5-tri-O-3-hydroxypropyl-myo-inositol-1,2,6-trisphosphate, D-3,4,5-tri-O-(6-hydroxy)-hexyl-myo-inositol-1,2,6-trisphosphate, D-5-O-hexyl-myo-inositol-1,2,6-trisphosphate, D-3,4,5-tri-O-phenylcarbamoyl-myo-inositol-1,2,6-trisphosphate, D-3,4,5-tri-O-propyl-myo-inositol-1,2,6-tris(carboxymethylphosphate), D-3,4,5-tri-O-butyl-myo-inositol-1,2,6-tris(carboxymethylphosphate), D-3,4,5-tri-O-isobutyl-myo-inositol-1,2,6-tris(carboxymethyl-phosphate), D-3,4,5-tri-O-pentyl-myo-inositol-1,2,6-tris(carboxymethylphosphate), D-3,4,5-tri-O-hexyl-myo-inositol-1,2,6-tris(carboxymethylphosphate), D-3,4,5-tri-O-propyl-myo-inositol-1,2,6-tris(carboxymethylphosphonate), D-3,4,5-tri-O-butyl-myo-inositol-1,2,6-tris(carboxymethyl-phosphonate), D-3,4,5-tri-O-isobutyl-myo-inositol-1,2,6-tris(carboxymethylphosphonate), D-3,4,5-tri-O-pentyl-myo-inositol-1,2,6-tris(carboxymethylphosphonate), D-3,4,5-tri-O-hexyl-myo-inositol-1,2,6-tris(carboxymethylphosphonate), D-3,4,5-tri-O-propyl-myo-inositol-1,2,6-tris(hydroxymethyl-phosphonate), D-3,4,5-tri-O-butyl-myo-inositol-1,2,6-tris(hydroxymethylphosphonate), D-3,4,5-tri-O-isobutyl-myo-inositol-1,2,6-tris(hydroxymethylphosphonate), D,-3,4,5-tri-O-pentyl-myo-inositol-1,2,6-tris(hydroxymethylphosphonate), D-3,4,5-tri-O-hexyl-myo-inositol-1,2,6-tris(hydroxymethyl-phosphonate), D-3,4,5-tri-O-propanoyl-myo-inositol-1,2,6-tris(carboxymethylphosphate), D-3,4,5-tri-O-butanoyl-myo-inositol-1,2,6-tris(carboxymethylphosphate), D-3,4,5-tri-O-isobutanoyl-myo-inositol-1,2,6-tris(carboxymethyl-phosphate), D-3,4,5-tri-O-pentanoyl-myo-inositol-1,2,6-tris(carboxymethylphosphate), D-3,4,5-tri-O-hexanoyl-myo-inositol-1,2,6-tris(carboxymethylphosphate), D-3,4,5-tri-O-propanoyl-myo-inositol-1,2,6-tris(carboxymethylphosphonate), D-3,4,5-tri-O-butanoyl-myo-inositol-1,2,6-tris(carboxymethyl-phosphonate), D-3,4,5-tri-O-isobutanoyl-myo-inositol-1,2,6-tris(carboxymethylphosphonate), D-3,4,5-tri-O-pentanoyl-myo-inositol-1,2,6-tris(carboxymethylphosphonate), D-3,4,5-tri-O-hexanoyl-myo-inositol-1,2,6-tris(carboxymethylphosphonate), D-3,4,5-tri-O-propanoyl-myo-inositol-1,2,6-tris(hydroxymethyl-phosphonate), D-3,4,5-tri-O-butanoyl-myo-inositol-1,2,6-tris(hydroxymethylphosphonate), D-3,4,5-tri-O-isobutanoyl-myo-inositol-1,2,6-tris(hydroxymethylphosphonate), D,-3,4,5-tri-O-pentanoyl-myo-inositol-1,2,6-tris(hydroxymethylphosphonate), and D-3,4,5-tri-O-hexanoyl-myo-inositol-1,2,6-tris(hydroxymethyl-phosphonate).

Inositol triphosphate is a preferred compound of the invention. When the compound of the invention is inositol triphosphate, preferred compounds are myo-inositol-1,2,6-trisphosphate and myo-inositol-1,2,3-trisphosphate, in particular in the form of a sodium salt. Particularly, the penta sodium salt of 1,2,6-D-myo inositol trisphosphate (Na₅H 1,2,6-D-myo-inositol trisphosphate), Mg₃ 1,2,6-D-myo-inositol trisphosphate or Ca₃ 1,2,6-D-myo-inositol trisphosphate).

When the cyclic moiety is a saccharide it is preferably selected from D/L-ribose, D/L-arabinose, D/L-xylose, D/L-lyxose, D/L-allose, D/L-altrose, D/L-glucose, D/L-mannose, D/L-gulose, D/L-idose, D/L-galactose, D/L-talose, D/L-ribulose, D/L-xylulose, D/L-psicose, D/L-sorbose, D/L-tagatose, D/L rhamnose and D/L-fructose, including derivatives thereof. Preferably the compound of the invention is a phosphate, a phosphonate or a phosphinate of a saccharide. Preferably the number of phosphate, phosphonate or phosphinate radicals per saccharide moiety is three or more. One or more of the hydroxyl groups on the saccharide moiety not bound to phosphorous can be etherified or esterified. Estherification and etherification is particularly preferred since it increases stability and prolongs half-life of the compound of the invention in vivo by reducing susceptibility to enzymatic degradation.

Preferred compounds having a saccharide moiety selected from mannose-2,3,4-trisphosphate, galactose-2,3,4-trisphosphate, fructose-2,3,4-trisphosphate, and altrose-2,3,4-trisphosphate and rhamnose-2,3,4-trisphosphate. Most preferred is to select the compound from R¹-6-O—R²-α-D-manno-pyranoside-2,3,4-trisphosphate, R¹-6-O—R²-α-D-galacto-pyranoside-2,3,4-trisphosphate, R¹-6-O—R²-α-D-altropyranoside-2,3,4-trisphosphate and R¹-6-O—R²-β-D-fructopyranoside-2,3,4-trisphosphate, wherein R¹ and R² independent of each other are defined as above, and preferably are methyl, ethyl, propyl, butyl, pentyl, or hexyl.

Preferred compounds of the invention comprising a saccharide moiety in which R¹ and/or R² are substituted in the aforementioned manner are selected from methyl-6-O-butyl-α-D-mannopyranoside-2,3,4-trisphosphate, methyl-6-O-butyl-α-D-galactopyranoside-2,3,4-trisphosphate, methyl-6-O-butyl-α-D-glycopyranoside-2,3,4-trisphosphate, methyl-6-O-butyl-α-D-altropyranoside-2,3,4-trisphosphate, methyl-6-O-butyl-β-D-fructopyranoside-2,3,4-trisphosphate, 1,5-anhydro-D-arabinitol-2,3,4-trisphosphate, 1,5-anhydroxylitol-2,3,4-trisphosphate, 1,2-O-ethylene-β-D-fructopyranoside-2,3,4-trisphosphate, methyl-α-D-rhamno-pyranoside-2,3,4-trisphosphate, methyl-α-D-mannopyranoside-2,3,4-trisphosphate, methyl-6-O-butyl-α-D-mannopyranoside-2,3,4-tris-(carboxy-methylphosphate), methyl-6-O-butyl-α-D manno-pyranoside-2,3,4-tris(carboxymethylphosphonate), methyl-6-O-butyl-α-D-manno-pyranoside-2,3,4-tris(hydroxymethyl-phosphonate), methyl-6-O-butyl-α-D-galactopyranoside-2,3,4-tris(carboxymethyl-phosphate), methyl-6-O-butyl-α-D-galacto-pyranoside-2,3,4-tris(carboxymethylphosphonate), methyl-6-O-butyl-α-D-galacto-pyranoside-2,3,4-tris(hydroxymethyl-phosphonate), methyl-6-O-butyl-α-D-glucopyranoside-2,3,4-tris(carboxymethylphosphate), methyl-6-O-butyl-α-D-glucopyranoside-2,3,4-tris(carboxymethyl-phosphonate), methyl-6-O-butyl-α-D-glucopyranoside-2,3,4-tris(hydroxymethyl-phosphonate), methyl-6-O-butyl-α-D-altropyranoside-2,3,4-tris-(carboxymethylphosphate), methyl-6-O-butyl-α-D-altropyranoside-2,3,4-tris-(carboxymethyl-phosphonate), methyl-6-O-butyl-α-D-altropyranoside-2,3,4-tris-(hydroxymethylphosphonate), methyl-6-O-butyl-β-D-fructo-pyranoside-2,3,4-tris-(carboxymethylphosphate), methyl-6-O-butyl-β-D-fructopyranoside-2,3,4-tris-(carboxymethyl-phosphonate), and methyl-6-O-butyl-β-D-fructo-pyranoside-2,3,4-tris-(hydroxymethylphosphonate).

When the cyclic moiety is an arabinitol, the compound of the invention is preferably a phosphate, phosphonate or phosphinate of arabinitol. Preferred arabinitol compounds, comprising a heterocyclic moiety, are selected from 1,5-dideoxy-1,5-iminoarabinitol-2,3,4-trisphosphate, 1,5-dideoxy-1,5-iminoarabinitol-2,3,4-tris-(carboxymethylphosphate), 1,5-dideoxy-1,5-imino-arabinitol-2,3,4-tris(carboxymethyl-phosphonate), 1,5-dideoxy-1,5-iminoarabinitol-2,3,4-tris(hydroxymethylphosphonate), 1,5-dideoxy-1,5-imino-N-(2-phenylethyl)arabinitol-2,3,4-trisphosphate, 1,5-dideoxy-1,5-imino-N-(2-phenylethyl)-arabinitol-2,3,4-tris(carboxymethyl-phosphate), 1,5-dideoxy-1,5-imino-N-(2-phenylethyl)arabinitol-2,3,4-tris-(carboxy-methylphosphonate), and 1,5-dideoxy-1,5-imino-N-(2-phenyl-ethyl)arabinitol-2,3,4-tris(hydroxymethylphosphonate).

When the cyclic moiety comprises one or more hydroxyl groups not bound to phosphorous-containing radicals at least one of said hydroxyl groups can be derivatized in the form of an ether or an ester. Esterification and etherification are preferred since there is an increase in stability and prolongation of half-life of this type of compounds in vivo due to reduced susceptibility to enzymatic degradation.

At least one of the hydroxyl groups of the cyclic moiety not bound to phosphorous-containing radicals can be derivatized to form an ester having the general formula IV

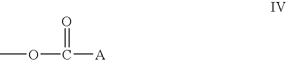

According to a first alternative A is a straight or branched saturated or unsaturated alkyl residue containing 1 to 24 carbon atoms to be selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methylheptyl, 2-methyloctyl, 2-methylnonyl, 2-methyldecyl, 2-methyleicosyl, 2-ethylbutyl, 2-ethylpentyl, 2-ethylhexyl, 2-ethylheptyl, 2-ethyloctyl, 2-ethylnonyl, 2-ethyldecyl, 2-ethyleicosyl, tert-butyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl and doeicosynyl.

According to a second alternative A is a saturated or unsaturated aromatic or non-aromatic homo- or heterocyclic residue or substituent to be selected from the group consisting of cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl and carbohydrate.

According to a third alternative A is (CH₂)ₙOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to a fourth alternative A is (CH₂)ₙZ(CH₂)ₘOR¹² where n and m is an integer between 1 and 10, where Z is oxygen or sulphur and where R¹² is hydrogen, a substituted or unsubstituted straight or branched alkyl, cycloalkyl, aryl or alkaryl; preferably n is 1, m is between 2 and 4 and R¹² is hydrogen or a lower alkyl such as methyl, ethyl or propyl.

According to a fifth alternative A is (CH₂)ₙOCOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to a sixth alternative A is (CH₂)ₙCOOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to a seventh alternative A is (CH₂)ₙOCOOR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen or a lower alkyl such as methyl, ethyl or propyl.

According to an eight alternative A is (CH₂)ₙR¹² where n is an integer between 1 and 10 and where R¹² is hydrogen, a substituted or unsubstituted straight or branched alkyl, cycloalkyl, aryl or alkaryl; preferably n is between 2 and 4 and R¹² is hydrogen, a lower alkyl such as methyl, ethyl or propyl.

According to a ninth alternative A is (CH₂)ₙOCONR¹²R¹³ where n is an integer between 1 and 10 and where R¹² and R¹³ are hydrogen, a substituted or unsubstituted straight or branched alkyl, cycloalkyl, aryl or alkaryl; preferably n is between 2 and 4 and R¹² and R¹³ are hydrogen or a lower alkyl such as methyl, ethyl or propyl.

The substituent A could be the same at all of the positions or could have different structures following the above definitions.

When the cyclic moiety is an inositol, triesters of the compounds are preferred. Most preferred compounds are selected from the group consisting of tri-O-hexanoyl-inositol-trisphosphate, tri-O-butanoyl-inositol-trisphosphate, tri-O-pentanoyl-inositol-trisphosphate, tri-O-(4-hydroxy)pentanoyl-inositol-trisphosphate, tri-O-isobutanoyl-inositol-trisphosphate, tri-O-propanoyl-inositol-trisphosphate, tri-O-(6-hydroxy-4-oxa)hexanoyl-inositol-trisphosphate, tri-O-3-(ethylsulfonyl)propanoyl-inositol-trisphosphate, tri-O-3-hydroxypropanoyl-inositol-trisphosphate, tri-O-(6-hydroxy)-hexanoyl-inositol-trisphosphate, tri-O-phenylcarbamoyl-inositol-trisphosphate, tri-O-dodecanoyl-inositol-trisphosphate, tri-O-(2-acetoxy)benzoylcarbamoyl-inositol-trisphosphate, tri-O-butylcarbamoyl-inositol-trisphosphate, tri-O-methylcarbamoyl-inositol-trisphosphate, and tri-O-phenylcarbamoyl-inositol-trisphosphate.

When the cyclic moiety is a myo-inositol, triesters of the compounds are preferred. Most preferred compounds are selected from the group consisting of D-3,4,5-tri-O-hexanoyl-myo-inositol-1,2,6-trisphosphate, D-3,4,5-tri-O-butanoyl-myo-inositol-1,2,6-trisphosphate, D-3,4,5-tri-O-pentanoyl-myo-inositol-1,2,6-trisphosphate, D-3,4,5-tri-O-(4-hydroxy)pentanoyl-myo-inositol-1,2,6-trisphosphate, D-3,4,5-tri-O-isobutanoyl-myo-inositol-1,2,6-trisphosphate, D-3,4,5-tri-O-propanoyl-myo-inositol-1,2,6-trisphosphate, D-3,4,5-tri-O-(6-hydroxy-4-oxa)hexanoyl-myo-inositol-1,2,6-trisphosphate, D-3,4,5-tri-O-3-(ethylsulfonyl)propanoyl-myo-inositol-1,2,6-trisphosphate, D-3,4,5-tri-O-3-hydroxypropanoyl-myo-inositol-1,2,6-trisphosphate, D-3,4,5-tri-O-(6-hydroxy)-hexanoyl-myo-inositol-1,2,6-trisphosphate, D-3,4,5-tri-O-phenylcarbamoyl-myo-inositol-1,2,6-trisphosphate, D-3,4,5-tri-O-dodecanoyl-myo-inositol-1,2,6-trisphosphate, D-3,4,5-tri-O-(2-acetoxy)benzoylcarbamoyl-myo-inositol-1,2,6-trisphosphate, D-3,4,5-tri-O-butylcarbamoyl-myo-inositol-1,2,6-trisphosphate, D-3,4,5-tri-O-methylcarbamoyl-myo-inositol-1,2,6-trisphosphate, and D-3,4,5-tri-O-phenylcarbamoyl-myo-inositol-1,2,6-trisphosphate in particular, in the form of their sodium salts. One preferred triester of the compound is D-3,4,5-tri-O-hexanoyl-myo-inositol-1-2,6-trisphosphate in the form of the penta sodium salt.

1,2,6-D-myo-inositol trisphosphate is formed from phytic acid by controlled enzymatic cleavage. 1,2,6-D-myo-inositol trisphosphate is stable in form of its salts that form aqueous solutions near the neutral point. If not otherwise indicated 1,2,6-D-myo-inositol trisphosphate is presumed to be present in such salt form. 1,2,6-D-myo-inositol trisphosphate in form of its salts and pharmaceutical compositions comprising 1,2,6-D-myo-inositol trisphosphate in form of its salts are disclosed in U.S. Pat. No. 4,777,134 A and U.S. Pat. No. 4,735,936 A, respectively. 1,2,6-D-myo-inositol trisphosphate is disclosed to have preventive effect in cardiovascular disease, cerebral disease, diseases of the respiratory system, diseases related to abnormal hormone release (U.S. Pat. No. 5,128,332 A), and other conditions in which neuropeptide Y is said to be involved. 1,2,6-D-myo-inositol trisphosphate does not pass through the cell membrane.

Pharmaceutically acceptable salts, in particular sodium, potassium, calcium and magnesium salts, of the compounds used according to the invention are also comprised by the invention. Particularly preferred is the penta sodium salt of 1,2,6-D-myo-inositol trisphosphate (Na₅H 1,2,6-D-myo-inositol trisphosphate) or another pharmaceutically acceptable salts of 1,2,6-D-myo-inositol trisphosphate, in particular the magnesium salt and the calcium salt.

According to a preferred aspect of the invention one or more, in particular all, of the hydroxyl groups in positions 3, 4, and 5 of 1,2,6-D-myo-inositol trisphosphate are esterified, such as with C₂-C₁₀ carboxylic acid, more preferred with saturated C₂-C₁₀ carboxylic acid, even more preferred with saturated and straight-chain C₂-C₁₀ carboxylic acid, most preferred with butyric acid, valeric acid and, in particular, caproic acid.

A preferred triester of the compound is 1D-3,4,5-trishexanoyl-myo-inositol-1-2,6-trisphosphate, in particular including in form of its penta sodium salt.

A pharmacologically effective amount of the compound of the invention is an amount that prevents, dampens and even stops the catabolism, in particular an amount that reduces or stops the rate of loss of lean muscle mass.

The present invention also discloses a method for inhibiting protein degradation and stimulating protein synthesis in catabolic patients, in particular cachectic patients.

In general, the compound according to the invention is administered in form of one of its pharmaceutically acceptable salts, in particular its sodium salt. Other compounds of the invention are preferably administered in a corresponding manner. In the following, a reference to alpha-trinositol comprises reference to the pharmaceutically acceptable salts of 1,2,6-D-myo-inositol trisphosphate, in particular the penta sodium salt.

Preferably the compounds according to the invention is used in essentially pure form, but its use in a purity of 80% or more, preferably of 90% or more, most preferred of 95% or more, is also comprised by the invention. Impurities accompanying the inositol trisphosphates used and administered according to the invention comprise or substantially consist of other pharmaceutically acceptable inositol phosphates. In particular, if the compound is the penta sodium salt of 1,2,6-D-myo inositol trisphosphate, the impurities comprise or substantially consist of other pharmaceutically acceptable inositol phosphates.

The compounds to be used/administered according to the invention can for example be administered intravenously. When administered intravenously, a preferred amount of alpha-trinositol is given to an adult person as a bolus injection from about 5 mg/kg body weight to about 80 mg/kg body weight, preferably about 10 mg/kg body weight to about 60 mg/kg body weight, more preferably from about 20 mg/kg or about 30 mg/kg to about 50 mg/kg, most preferred about 40 mg/kg. It is preferred to administer alpha-trinositolintravenously at a rate to maintain the plasma level thereof at or near the maximum plasma level obtained by injecting a bolus of alpha-trinositol of from about 5 mg/kg body weight to about 80 mg/kg body weight, about 10 mg/kg to about 60 mg/kg, more preferred from about 20 mg/kg or about 30 mg/kg to about 50 mg/kg, most preferred about 40 mg/kg. Alternatively, the administration of two or more separate intravenous bolus injections over a day spaced by from 1 to 12 hrs of the compound of from about 5 mg/kg body weight to about 80 mg/kg body weight, about 10 mg/kg to about 60 mg/kg of the compound is preferred, more preferred of from about 20 mg/kg or about 30 mg/kg to about 50 mg/kg, most preferred of about 40 mg/kg.

If the compound to be used/administered is an ester as described above, such as an ester of alpha-trinositol, the amount administered is about 0.1 mg/kg body weight to about 20 mg/kg body weight, preferably about 1 mg/kg to about 10 mg/kg and more preferably about 4 mg/kg to about 8 mg/kg.

The administration of alpha-trinositol according to the invention to a patient afflicted with a catabolic condition or a patient at risk of developing a catabolic condition can proceed as long as there is manifest cachexia or a risk of cachexia, such as over a period of from one day to a week or two weeks and even for a month of more. Due to the nature of alpha-trinositol such treatment is well tolerated. Preferred administration ranges (mg of compound of the invention/kg body weight) for other compounds of the invention can be easily determined by titration of animal models and/or patients with catabolic disorders.

Alternatively, alpha-trinositol or other compounds according to the invention, including their pharmaceutically acceptable salts, is administered subcutaneously or intramuscularly.

It is also within the scope of the invention to provide an adequate plasma level of alpha-trinositol or other compounds according to the invention in a patient by means of an implant, such as an infusion pump, which may be implanted and designed for slow release.

According to the invention it is also disclosed a pharmaceutical composition comprising the compounds as described above. The composition can be adapted for intravenous administration, including intravenous bolus injection and intravenous infusion over an extended period of time, such as for hours and even a day or more, comprising a pharmacologically effective amount of alpha-trinositol or other compounds according to the invention and an aqueous solvent, in particular saline, and a pharmaceutically acceptable carrier. Preferably such composition is in a closed container and in crystalline or amorphous form, including in form of a cryoprecipitate. The composition can also be dispersed in a stabilizing agent or a mixture of stabilizing agents, in particular in one or more of glucose, mannose, sodium chloride.

According to another preferred aspect of the invention the composition for intravenous infusion additionally comprises an analgesic agent, in particular an opoid agonist. It is preferred for the opoid agonist to be selected from morphine, nalorphine, nalbuphine, levorphanol, racemorphan, levallorphan, dextromethorphan, cyclorphan, butorphanol, pentazocine, phenazocine, cyclazocine, ketazocine, pethidine, meperidine diphenoxylate, anileridine, piminodine, fentanil, ethoheptazine, alphaprodine, betaprodine, 1-methyl-4-phenyl-1,2,5,6-tetrahydropyridine (MPTP), loperamide, sulfentanil, alfentanil, remifentanil, lofentanil, methadone, d-propoxyphene, isomethandone, levo-alpha-acetylmethadol (LAAM), naloxone, naltrexone, natrindole, oripavine and its derivatives, codeine, heterocodeine, morphinone, dihydromorphine, dihydrocodeine, dihydromorphinone, dihydrocodeinone, 6-desoxumorphine, oxymorphone, oxycodone, 6-methylene-dihydromorphine, hydrocodone, hydromorphone, metopon, apomorphine, normorphine, N-(2-phenylethyl)-normorphine, etorphine, buprenorphine, spiradoline, enadoline or asimadoline.

Further according to a particular embodiment of the invention, an effective amount of the previously recited compounds generally described in the foregoing to comprise a high density, negatively charged domain of vicinally oriented radicals, preferably capable of complexing divalent cations can be combined with nutrients with the purpose of treating cachexia or other serious forms of catabolism associated with severe trauma or other conditions which frequently are difficult or impossible to reverse with conventional nutritional regimens. Such catabolic conditions may for example be induced from sepsis and severe burns or other catabolic conditions as specified above.

A therapy including the recited type of compounds and the nutrients can be an adjunct therapy wherein the components are administered separately according to suitable predetermined schemes, or it can be a co-administration in a form suitable or conventional for administering parenteral or enteral nutrients. Numerous products for nutrition in critical care are available and they are commonly based on one or several of lipid emulsions, sources of amino acids and carbohydrates (sugars). Especially for parenteral nutrition, products are developed with special consideration to compatibility of the ingredients during terminal sterilization and long-term storage. The persons skilled in this technology are also aware of nutritional constituents with documented usefulness in catabolic conditions such as omega-3-fatty acids (from an oil source) and branched chain amino acids (e.g. valine, leucine and isoleucin).

The combination therapy with selected nutrients according to the present invention aims at further enhancing the treatment of the severely catabolic patients by replenishing depleted supplies of nutrients in the skeletal muscles and restore their overall body mass. According to one aspect of the invention a nutritional composition is provided comprising an inositol triphosphate or an ester thereof, or a mono- or disaccharide having three or more phosphate radicals per saccharide moiety or an ester thereof; and at least one nutrient selected from the group consisting of lipid emulsions, fluid sources of amino acids and carbohydrates.

When the composition is adapted to parenteral administration it comprises suitably manufactured constituents in a vehicle suitable for this administration route. A nutritional composition for oral or enteral administration may include taste enhancers and conventional ingredients well know for practitioners in this field. Examples of suitable nutrients are fluid sources of amino acids or conjugates or precursors thereof (e.g. peptides), lipid emulsions comprising oil phases with long- or medium chain fatty acids and carbohydrate solutions (comprising glucose and/or other energy rich compounds).

The nutrient compositions may further comprise constituents well-know in the field such as vitamins, trace elements, electrolytes, isotonicty adjusters and the like, as well complementary drugs dependent on the clinical situation.

In another embodiment the invention relates to the use of an inositol triphosphate or an ester thereof, or a mono- or disaccharide having three or more phosphate radicals per saccharide moiety or an ester thereof; and at least one nutrient selected from the group consisting of lipid emulsions, fluid sources of amino acids and carbohydrates for the preparation of a nutritional supplement for preventing and/or treating cachexia or catabolic conditions associated with severe trauma.

Preferably, the inositol triphosphate or an ester thereof, or a mono- or disaccharide having three or more phosphate radicals per saccharide moiety or an ester thereof is supplied to a composition adapted for parenteral administration just its before administration. In one embodiment, such composition comprises a solution of carbohydrates.

The use of the nutritional supplement provides about 5 to about 80, preferably about 10 to about 60 mg per kg body weight of inositol triphosphate or an ester thereof, or a mono- or disaccharide having three or phosphate radicals per saccharide moiety or an ester thereof to a patient.

The invention will be described in more detail by reference to a number of preferred embodiments illustrated by figures.

### DESCRIPTION OF THE FIGURES

FIG. 1 is a diagram showing the change in body weight in mice bearing the MAC16 tumour treated with alpha-trinositol (AT) in doses of 10 mg/kg, 20 mg/kg, and 40 mg/kg body weight, c=p<0.001 from control;

FIG. 2 is a diagram showing the corresponding change in tumour volume, b=p<0.01 from control;

FIG. 3 is a staple diagram showing the reduction in body weight in the model of FIG. 1 at a daily dosage of 3×40 mg/kg of alpha-trinositol;

FIG. 4 is a staple diagram showing the reduction in tumour volume in the model of FIG. 1 at three dosage (10 mg/kg, 20 mg/kg, and 40 mg/kg body weight) levels of alpha-trinositol (AT);

FIG. 5 is a diagram showing effect of PIF (proteolysis inducing factor) on protein degradation in murine myotubes in the presence of alpha-trinositol (AT, 100 μM). Differences from control are indicated as c, p<0.001, while differences in the presence of AT is shown as f, p<0.001.

FIG. 6 is a diagram showing the effect of 4.2 nM PIF on the chymotrypsin like activity in C2C12 myotubes in the presence the alpha-trinositol (AT, 100 μM). Difference from control is indicated as c, p<0.001.

FIG. 7 is a diagram showing the effect of PIF on the chymotrypsin like activity in C2C12 myotubes in the presence the lipid soluble derivative of alpha-trinositol (H)AT at 100 μM. Differences from control are indicated as c, p<0.001, while differences from alpha-trinositol (AT) are indicated as either e, p<0.01 or f, p<0.001.

FIG. 8 is a diagram showing the effect of Ang II on protein degradation in murine myotubes in the presence of alpha-trinositol (AT, 100 μM). Differences from control are indicated as c, p<0.001, while differences in the presence of AT is shown as f, p<0.001.

FIG. 9 is a diagram showing the effect of Angiotensin II induced chymotrypsin like activity in C2C12 myotubes in the presence of alpha-trinositol (AT, 100 μM).

FIG. 10 is a diagram showing the body weight change in MAC16 tumour-bearing mice treated with and without 40 mg/kg alpha-trinositol (AT). α—control; α—AT.

FIG. 11 is a staple diagram showing the gastrocnemius muscle weights in MAC16 tumour-bearing mice treated with and without 40 mg/kg alpha-trinositol (AT).

FIG. 12 is a staple diagram showing protein synthesis in the gastrocnemius muscle in MAC16 tumour-bearing mice treated with and without 40 mg/kg alpha-trinositol (AT).

FIG. 13 is a staple diagram showing protein degradation in the gastrocnemius muscle in MAC16 tumour-bearing mice treated with and without 40 mg/kg alpha-trinositol (AT).

FIG. 14 is a staple diagram showing the chymotrypsin activity in gastrocnemius muscle in MAC16 tumour-bearing mice treated with and without 40 mg/kg alpha-trinositol (AT).

FIG. 15 is a staple diagram showing the expression of the 20S proteasome subunit in gastrocnemius muscle in MAC16 tumour-bearing mice treated with and without 40 mg/kg alpha-trinositol (AT).

FIG. 16 is a staple diagram showing the P42 expression in gastrocnemius muscle in MAC16 tumour-bearing mice treated with and without 40 mg/kg alpha-trinositol (AT).

FIG. 17 is a staple diagram showing the myosin expression in gastrocnemius muscle in MAC16 tumour-bearing mice treated with and without 40 mg/kg alpha-trinositol (AT).

FIG. 18 is a staple diagram showing the ratio of phosphoPKR/total PKR in gastrocnemius muscle in MAC16 tumour-bearing mice treated with and without 40 mg/kg alpha-trinositol (AT).

FIG. 19 is a staple diagram showing the ratio of pelF2α/total elF2α in gastrocnemius muscle in MAC16 tumour-bearing mice treated with and without 40 mg/kg alpha-trinositol (AT).

FIG. 20 is a staple diagram showing the expression of mTOR in gastrocnemius muscle in MAC16 tumour-bearing mice treated with and without 40 mg/kg alpha-trinositol (AT).

FIG. 21 is a staple diagram showing the expression of 4E-BP1 in gastrocnemius muscle in MAC16 tumour-bearing mice treated with and without 40 mg/kg alpha-trinositol (AT).

FIG. 22 is a staple diagram showing the ratio of total 4E-BP1/toal elF4e in gastrocnemius muscle in MAC16 tumour-bearing mice treated with and without 40 mg/kg alpha-trinositol (AT).

FIG. 23 is a staple diagram showing the ratio of total eIF4G/total eIF4E in gastrocnemius muscle in MAC16 tumour-bearing mice treated with and without 40 mg/kg alpha-trinositol (AT).

FIG. 24 is a diagram showing the caspase 3 activity in gastrocnemius muscle in MAC16 tumour-bearing mice treated with and without 40 mg/kg alpha-trinositol (AT).

FIG. 25 is a diagram showing the caspase 8 activity in gastrocnemius muscle in MAC16 tumour-bearing mice treated with and without 40 mg/kg alpha-trinositol (AT).

FIG. 26 is a diagram showing the weight change in MAC16 tumour-bearing mice treated with lipid soluble alpha-trinositol (AT, 6 mg/kg and 8 mg/kg).

FIG. 27 is a diagram showing the food consumption in MAC16 tumour-bearing mice treated with lipid soluble alpha-trinositol (AT, 6 mg/kg and 8 mg/kg).

FIG. 28 is a diagram showing the water consumption in MAC16 tumour-bearing mice treated with lipid soluble alpha-trinositol (AT, 6 mg/kg and 8 mg/kg).

The present invention will now be further disclosed in the following non-limiting examples.

### EXAMPLES

### Example 1

### Treatment of Cachectic Mice with Alpha-Trinositol

Materials. Alpha-trinositol (1-D-Myo-inositol 1,2,6-triphosphate) was prepared according to U.S. Pat. No. 4,777,134. In a glass ampoule a stock solution was prepared by dissolving 1 g of alpha-trinositol in saline to a total volume of 10 ml. The stock solution was stored in a refrigerator for use within 24 hrs.

Animals. Pure strain male NMRI mice (average body weight 25 g) were transplanted with fragments of the MAC16 tumour subcutaneously into the flank by means of a trochar, selected from donor animals with established weight loss (Bibby M C et al., Characterization of transplantable adenocarcinoma in the mouse colon producing cachexia in the recipient animals. J Natl Cancer Inst 78 (1987) 539-546). Transplanted animals were given a rat and mouse breeding diet (Special Diet Services, Witham, UK) and water at lib. Weight loss was evident 10 to 12 days after tumour implantation. Just prior to the development of weight loss 24 animals were randomized into four groups (I-IV) of six animals each.

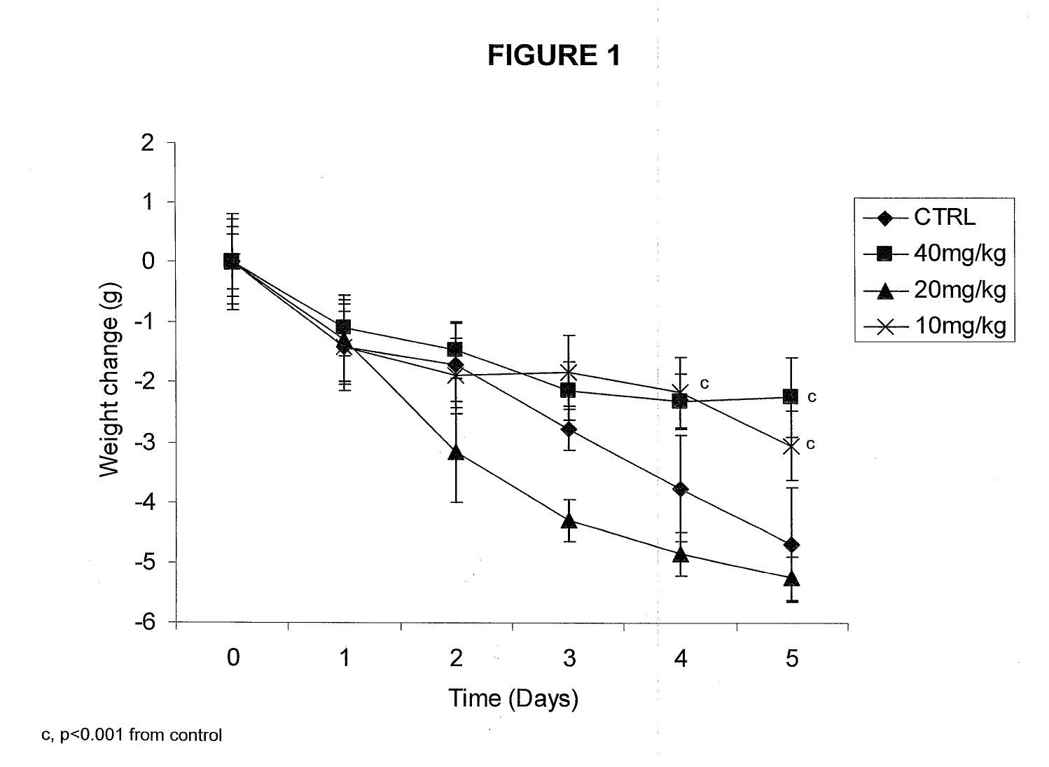

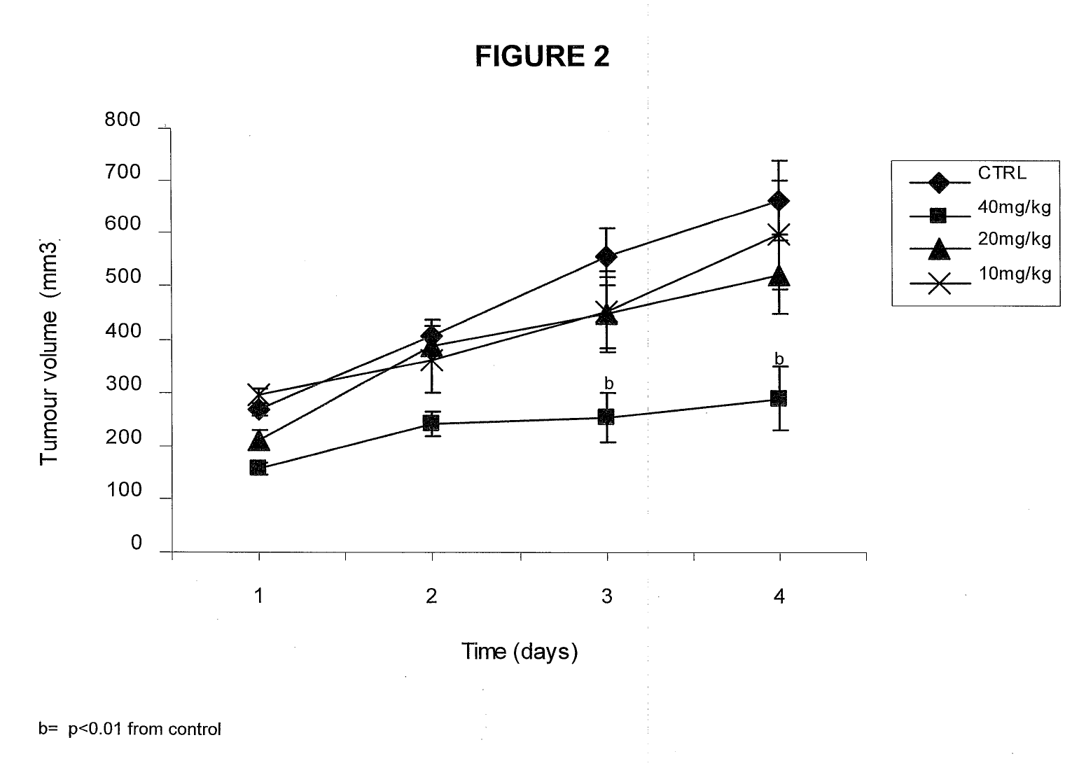

Administration of alpha-trinositol. A first experiment was started when the animals had lost about 5% of body weight. Aliquots (2.5 μl, Group I; 5 μl, Group II; 10 μl, Group III) of alpha-trinositol stock solution corresponding to doses of 10 mg/kg, 20 mg/kg, and 40 mg/kg for a 25 g animal were administered subcutaneously three times per day at 8.00, 12.00 and 16.00 hrs. The fourth group (Group IV; control) was given an intravenous injection of 10 μl of water. Body weight (FIG. 1), tumour volume (FIG. 2), and water and food intake were determined daily after the last injection.

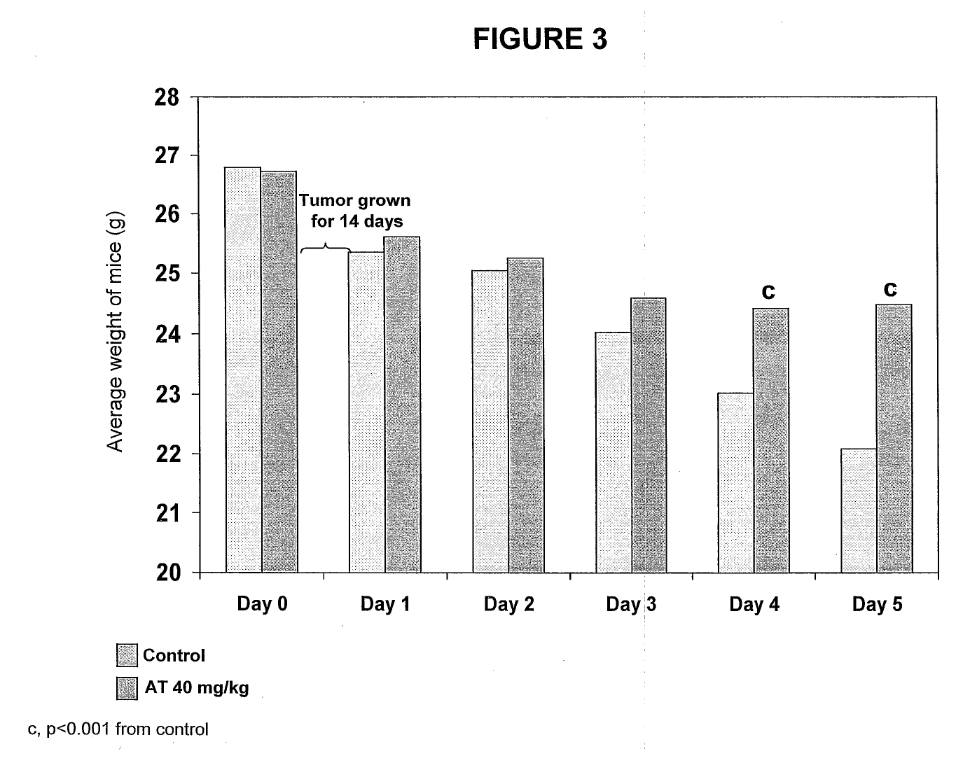

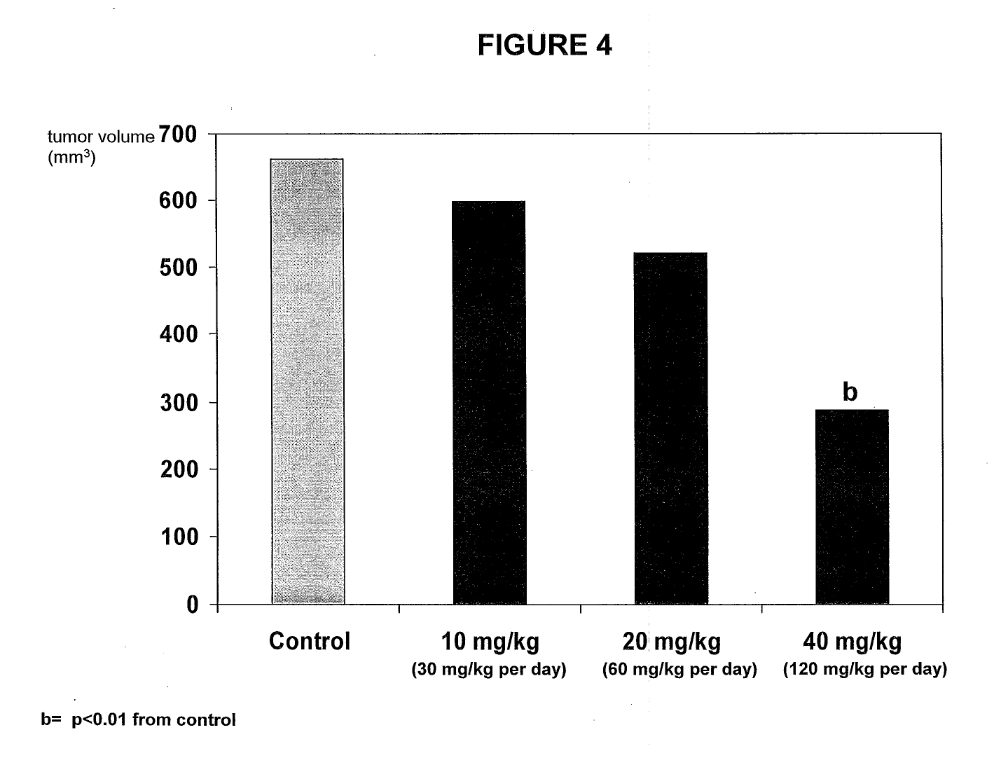

The reduction in body weight in the model of FIGS. 1 and 2, at the optimal daily dosage of 3×40 mg/kg of alpha-trinositol is shown in FIG. 3. FIG. 4 illustrates the reduction in tumour volume at the three alpha-trinositol dosage levels of the first experiment.

All groups lost the initial 1 gram from day 0 to day 1 (day 1 =2 weeks of tumour growth from day 0). This is to ascertain that all animals have developed cachexia. Thus, the difference in weight change is even more pronounced if a comparison is made from day 1 (i.e. the first day of treatment) to day 5 (the end of the treatment). From day 0 to day 5 the control group lost ˜4.7 g and the AT group ˜2.3 meaning that the relative weight loss in the AT group was half of the control group. However, from day 1 to day 5 the control group lost 3.3 g and AT group lost 1.15 g, giving a loss of ⅓ of the control group.

Interestingly, the dose of 10 mg/kg gave a clear anti-cachexia effect (as good as the 40 mg/kg, see FIG. 1). However, the dose of 10 mg/kg did not produce any statistically significant effect in terms of tumour inhibition. This suggests that the anti-cachexic effect is not caused by a tumour inhibitory effect, i.e. AT inhibits cachexia through a tumour independent pathway.

### Example 2

### Effect of Cachexia Treatment on Body Composition

At the end of the treatment described in Example 1 the mice were sacrificed, and their body composition was analyzed. The results are given in Table 1. They demonstrate that the method of the invention not only conserves the lean body mass in the animals but that it is even increased in relative as well as in absolute terms. The reduction of lean body mass is normally observed in cachectic patients and is a significant cause of morbidity. No significant change in water content was observed.

TABLE 1
Body composition (% by weight) of cachectic MAC16
mice

| alpha- | | | | | | |
|---|---|---|---|---|---|---|
| Trinositol | | | | | | |
| (mg/ml) | Water | p | Fat | p | Lean mass | p |
| 0 | 67.8 ± 1.2 | — | 6.1 ± 2.1 | — | 26.1 ± 1.6 | — |
| 10 | 68.0 ± 2.0 | NS | 4.9 ± 2.3 | NS | 27.1 ± 3.0 | NS |
| 20 | 68.1 ± 1.7 | NS | 3.3 ± 0.5 | 0.01 | 28.6 ± 1.7 | 0.05 |
| 40 | 65.6 ± 1.7 | NS | 3.7 ± 1.9 | 0.05 | 30.7 ± 2.5 | 0.01 |

Values are mean ± SD;
p values are from 0AT.

The absolute changes from controls are given in Table 2.

TABLE 2
Body composition of cachectic MAC16 mice,
absolute fat and lean mass changes

| alpha-Trinositol (mg/ml) | Fat (% by weight) | Lean mass (% by weight) |
|---|---|---|
| 10 | −19 | +11 |
| 20 | −46 | +15 |
| 40 | −39 | +25 |

### Example 3

### In Vitro Inhibition of PIF (Proteolysis Inducing Factor) and Angiotensin II with Alpha-Trinositol

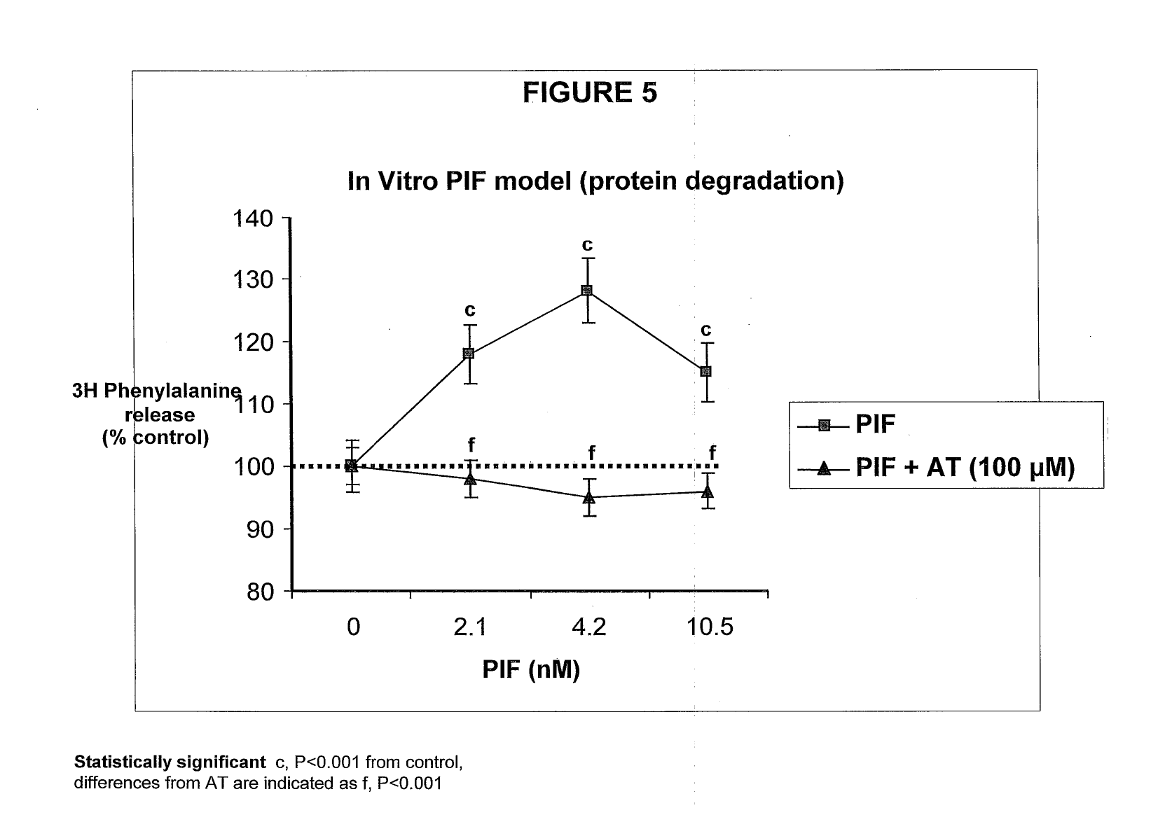

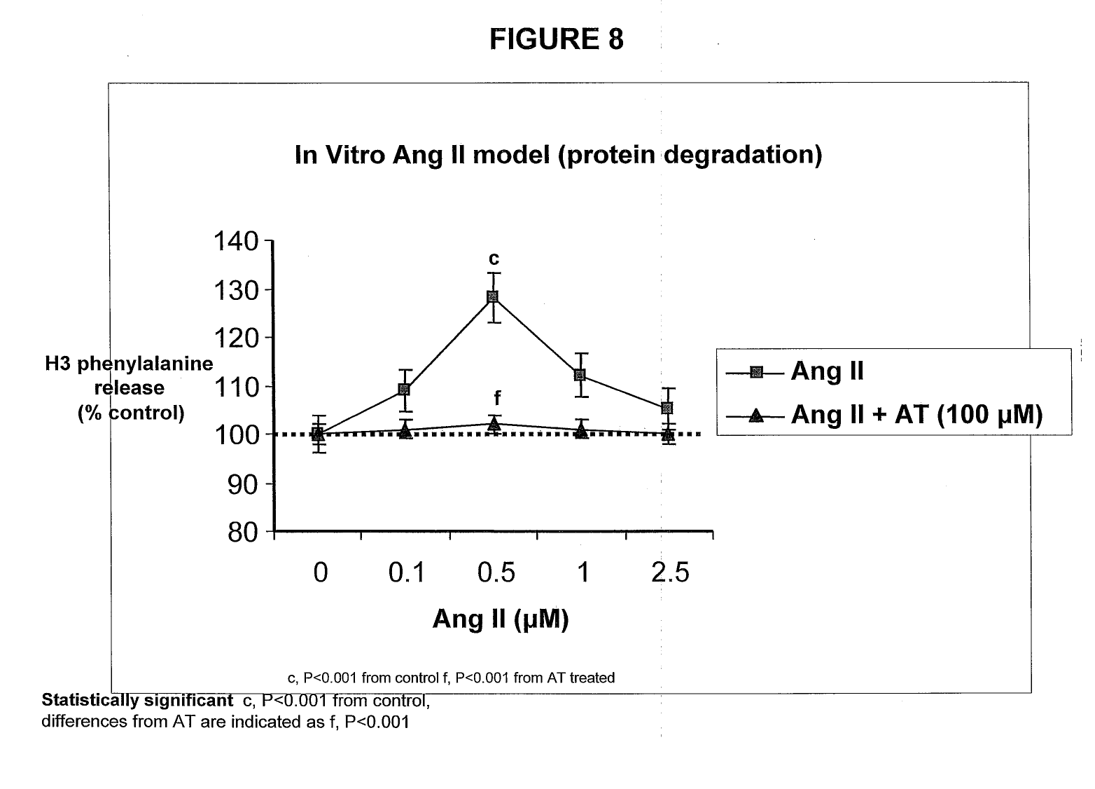

To investigate the mechanism by which AT protects lean body mass in cachexia, further experiments were carried out in vitro using murine myotubes as a surrogate model of skeletal muscle. Incubation with either PIF or angiotensin II (Ang II) induced protein degradation with a characteristic bell-shaped dose-response curve as previously reported (Smith et al., 2004, Br. J. Cancer, and Tisdale et al., 2006, Cell. Sig) with the maximal effect of PIF at 4.2 nM and Ang II at 0.5 μM (FIGS. 5 and 8). Incubation of myotubes with AT (100 μM) 2 h prior to addition of either PIF (FIG. 5) or Ang II (FIG. 8) completely attenuated protein degradation down to basal levels.

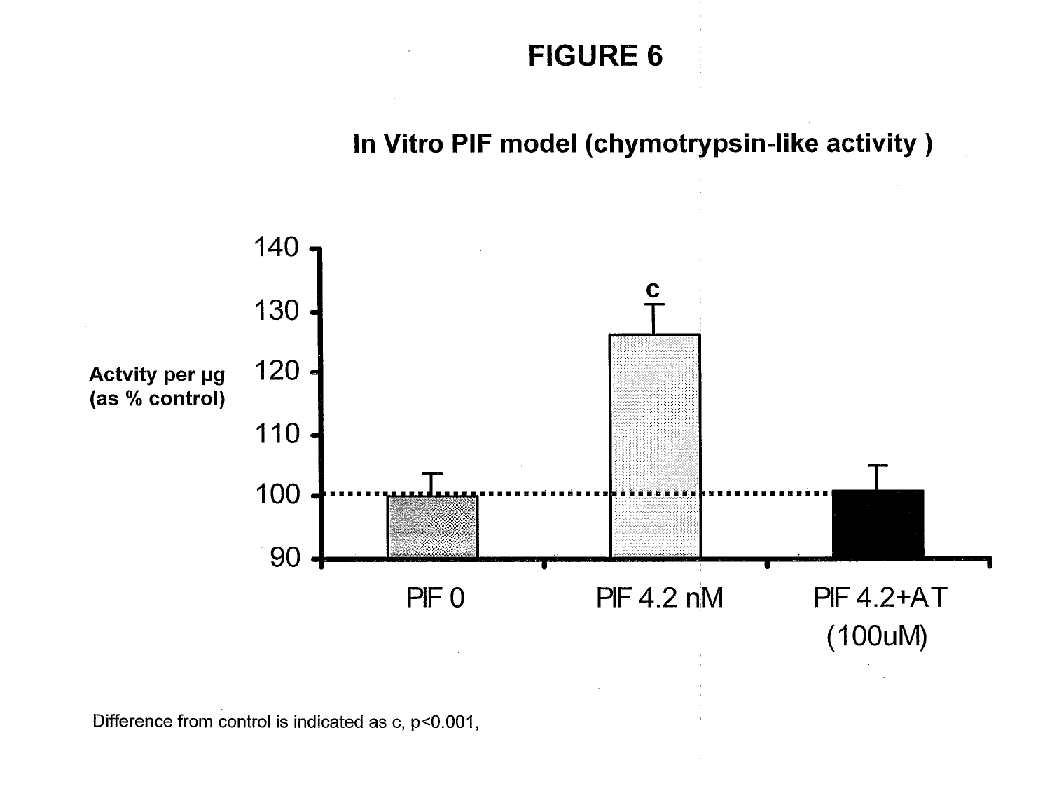

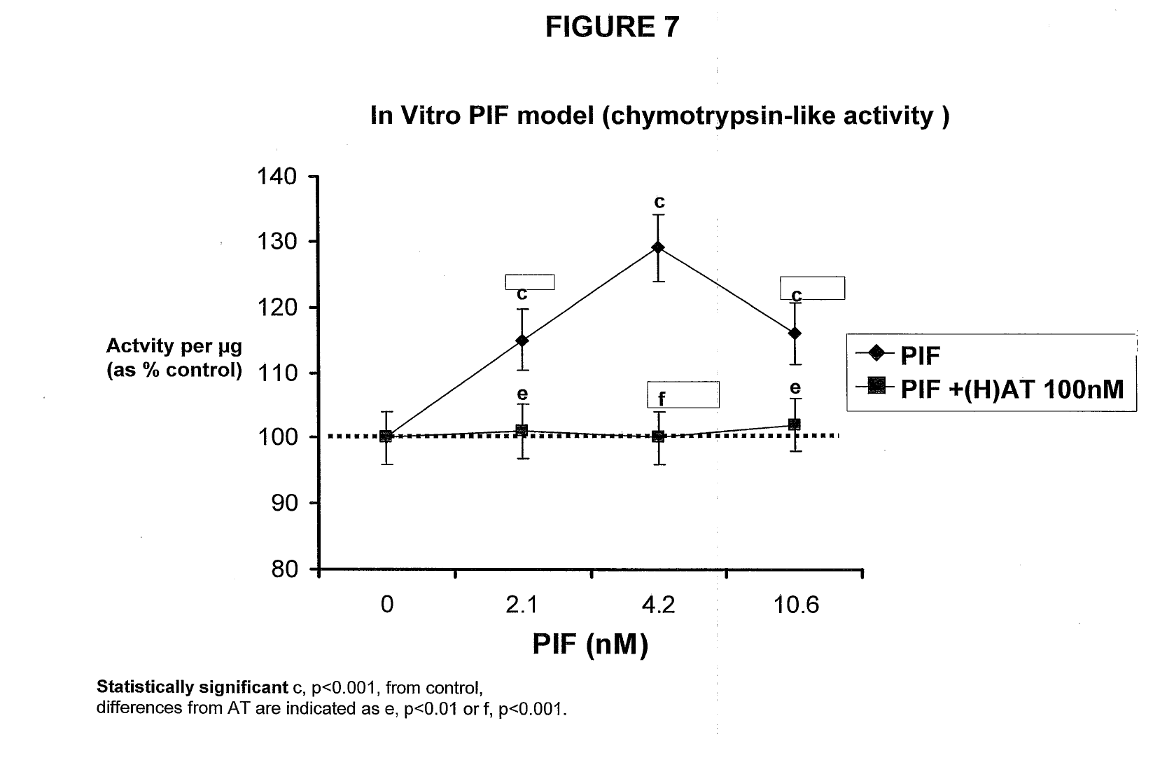

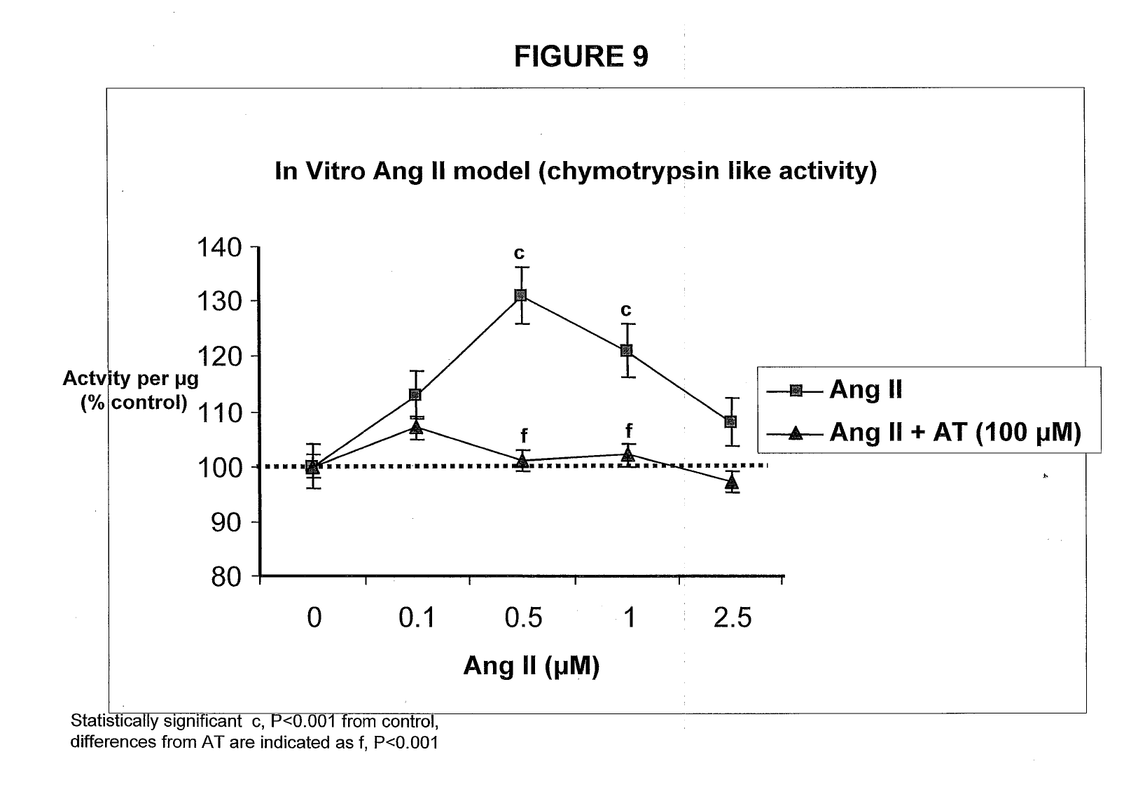

Protein degradation induced by PIF is mediated through up-regulation of the ubiquitin-proteasome pathway (Tisdale et al., 2004, Br. J. Cancer). Measurement of the chymotrypsin-like enzyme activity in myotubes, which is the major proteolytic activity of the β-subunits of the proteasome, showed an increase in the presence of 4.2 nM PIF (FIG. 6), and this effect was completely attenuated in the presence of AT (100 μM). A hexanoyl ester of AT (lipid soluble AT) has been produced as a slow release form, after hydrolysis by esterase. The results in FIG. 7 show that the lipid soluble derivative of AT (also a concentration of 100 mM), was as effective as the water soluble AT in attenuating the PIF-induced chymotrypsin-like enzyme activity. Measurement of the chymotrypsin-like enzyme activity in myotubes, showed an increase also in the presence of Ang II (FIG. 9). This effect was completely attenuated in the presence of AT 100 μM).

Chymase is a serine protease with chymotryptic activity and is one of the most abundant proteins in mast cell secretatory granules. Chymase is positively charged and binds heparin. (Takao et al Jpn J Pharmacol, 81, 1999, 404). Chymase is also suggested to have the same effect as angiotensin converting enzyme, i.e. to convert Ang I into Ang II. Thus, AT may also have an inhibitory effect against chymase and the destructive activity induced by chymase in various pathological situations.

### Example 4

### Effect on Protein Synthesis and Degradation in Muscles of MAC16 Tumour-Bearing Mice after Treatment with Alpha Trinositol

This study confirms the previous results that treatment of tumour-bearing mice with alpha-trinositol attenuates loss of body weight through preservation of lean body mass

Materials. Alpha-trinositol was prepared according to U.S. Pat. No. 4,777,134. In a glass ampoule a stock solution was prepared by dissolving 1 g of alpha-trinositol in saline to a total volume of 10 ml. The stock solution was stored in a refrigerator for use within 24 hrs.

L-[2,6-³H]Phenylalanine (sp.act.1.96TBq/mmole), Hybond A nitrocellulose membranes, m⁷GTP (7-methyl-GTP) Sepharose 4B, and ECL development kits were from Amersham Biosciences Ltd (Bucks, UK). Mouse monoclonal antibodies to 20S proteasome α-subunits and p42 were from Affiniti Research Products (Exeter, UK). Rabbit monoclonal antibodies to phospho-4EBP1 (Thr^{37/46}), phospho mTOR (Ser²⁴⁴⁸) and Thr⁵⁶ to phospho and total PKR, as well as rabbit polyclonal antisera to 4E-BP1, eIF4E, eIF4G, and to phospho and total elongation factor 2 (eEF2) were purchased from New England Biolabs (Herts, UK). Rabbit polyclonal antisera to phospho eIF2α (Ser⁵¹) and to total eIF2α was from Santa Cruz Biotechnology (CA). Rabbit polyclonal antisera to myosin heavy chain were from Novocastra (Newcastle, UK). Rabbit polyclonal antisera to mouse β-actin and the chymotrypsin substrate succinyl LLVY-7-amino-4-methylcoumarin were purchased from Sigma Aldridge (Dorset, UK). Peroxidase-conjugated rabbit anti-mouse antibody and peroxidase-conjugated goat anti-rabbit antibody were purchased from Dako Ltd (Cambridge, UK). Phosphosafe™ extraction reagent was from Merck Eurolab Ltd (Leicestershire, UK). The caspase-3 and -8 substrates and inhibitors were purchased from Biomol International (Devon, UK).

Animals. Pure strain male NMRI mice were transplanted with fragments of the MAC16 tumour as described in Example 4.

Administration of alpha-trinositol. Mice (n=6) bearing the MAC16 tumour were treated with AT (40 mg/kg) s.c., 3 times per day, for 5 days, while a control group received PBS. Protein synthesis and degradation were determined by the incorporation and release of L-[2,6-³H]phenylalanine, as described in Smith et al., Cancer Res., 2005; 65:277-83. On the fourth day of treatment half of the group administered 0.4 mmol/L L-[2,6-³H]phenylalanine in PBS (100 μl) by i.p. administration.

Protein analysis. After 24 h the animals were terminated and the gastrocnemius muscle was removed, washed with PBS and RPMI 1640, and the release of radioactivity during incubation for 2 h in RPMI 1640 was determined. Protein bound activity was determined by homogenising the muscles in 2% perchloric acid, and determining the radioactivity in the precipitate. Protein degradation was calculated by dividing the amount of radioactivity released into the medium over a 2 h period by the specific activity of the protein-bound radioactivity. To determine protein synthesis gastrocnemius muscles were incubated for 2 h in RMPI 1640, without phenol red, in the presence of L-[2,6-³H]phenylalanine (37 MBq), and under an atmosphere of O₂/CO₂ (19:1). Muscles were then rinsed in nonradioactive media, and homogenised in 2% perchloric acid. The rate of protein synthesis was calculated by dividing the protein-bound radioactivity by the acid-soluble material.

Determination of proteasome activity The activity of the 20S proteasome was determined as the ‘chymotrypsin-like’ enzyme activity, the predominant proteolytic activity of the β5 subunits of the proteasome. Gastrocnemius muscles were rinsed with ice-cold PBS and homogenised in 20 mmol/L Tris-HCl (pH 7.5), 2 mmol/L ATP, 5 mmol/L MgCl₂ and 1 mmol/L DTT followed by sonication. The sonicate was centrifuged for 10 minutes at 18,000×g at 4° C., and enzyme activity in the supernatant was determined by the method of Orino et al., FEBS Lett., 1991; 284:206-10, by determining the release of amino methyl coumarin (AMC) from the fluorogenic substrate LLVY-AMC. Activity was measured in the absence and presence of the specific proteasome inhibitor lactacystin (10 mmoles/L). Only lactacystin suppressible activity was considered to be proteasome specific.

Western blot analysis Gastrocnemius muscle (10 mg) was homogenised in Phosphosafe™ Extraction Reagent (500 μl) and centrifuged at 15000 g for 15 min. Samples of the cytosolic proteins (5 μg) were loaded on either a 10% (mTOR, myosin, eIF4E and eIF4G) 12% (PKR, eIF2α and actin) or 15% (4E-BP1) sodium dodecylsulphate-polyacrylamide gel (SDS-PAGE) and electrophoresed at 180 V for approximately 1 h. The extent of phosphorylation of 4E-BP1, and the association of 4E-BP1 and eIF4G with eIF4E was determined by Western blotting when eIF4E was extracted from the muscle samples by m⁷GTP-Sepharose 4B-affinity binding, as previously described Eley ey al., Biochem J., 2007; 407:113-20, by loading 20 μg of protein. The protein on the gels was then transferred to 0.45 mm nitrocellulose membranes, which were then blocked with 5% Marvel in Tris-buffered saline, pH 7.5, at 4° C. overnight. The primary antibodies were used at a dilution of 1:1000, except for phospho and total eIF2α (1:500) and myosin (1:250). The secondary antibodies were used at a dilution of 1:1000. Incubation was either for 1 h at room temperature, or overnight, and development was by ECL. Blots were scanned by a densitometer to quantify differences.

Determination of Caspase activity. The activity of caspase 3 was determined by the release of 7-amino-4-methylcoumarin (AMC) from the specific substrate AcDEVD-AMC in the presence or absence of the caspase 3 inhibitor AcDEVD-CHO. Muscle (10 mg) was homogenised in lysis buffer (150 mmol/L NaCl, 1% NP40, 50 mmol/L Tris HCl, pH 7.4, 0.25% sodium deoxycholate, 2 mmol/L EGTA, 1 mmol/L EDTA, 0.2 mmol/L sodium orthovanadate, 20 mmol/L NaF and 1% proteasome inhibitor mixture), left at 4° C. and then room temperature for 10 min, followed by centrifugation at 15,000 g for 15 min. The supernatant (50 μg protein) was incubated with the caspase 3 substrate for 1 h and the increase in fluorescence due to AMC was determined at an excilation wavelength of 370 nm and an emission wavelength of 430 nm. The difference in values in the absence and presence of the caspase-3 inhibitor was a measure of activity. The method for caspase 8 was similar with the substrate being Z-IETD-AFC and the inhibitor IETD-CHO. The increase in fluorescence due to the release of 7-amino-4-trifluoro-methylcoumarin (AFC) was measured with an excitation wavelength of 400 nm and an emission of 505 nm.

Statistical analysis. All results are shown as mean±S.E. for at least three replicate experiments. Differences in means between groups were determined by one-way analysis of variance followed by Tukey-Kramer multiple comparison test. p values less than 0.05 were considered ‘signifcant’.

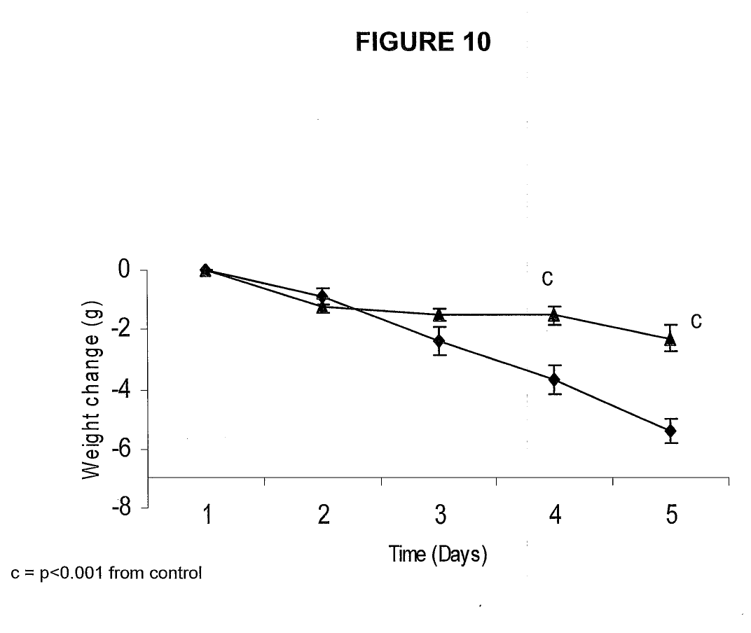

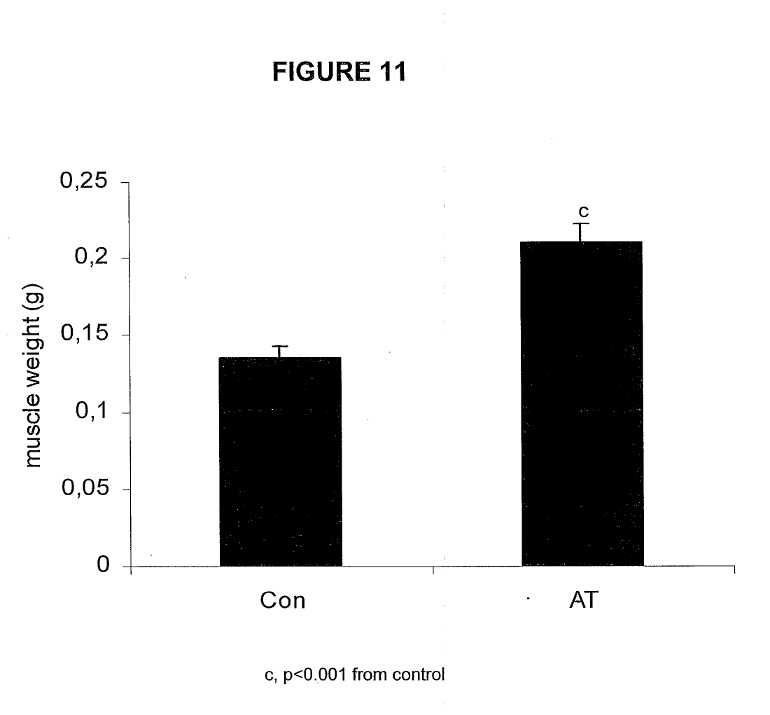

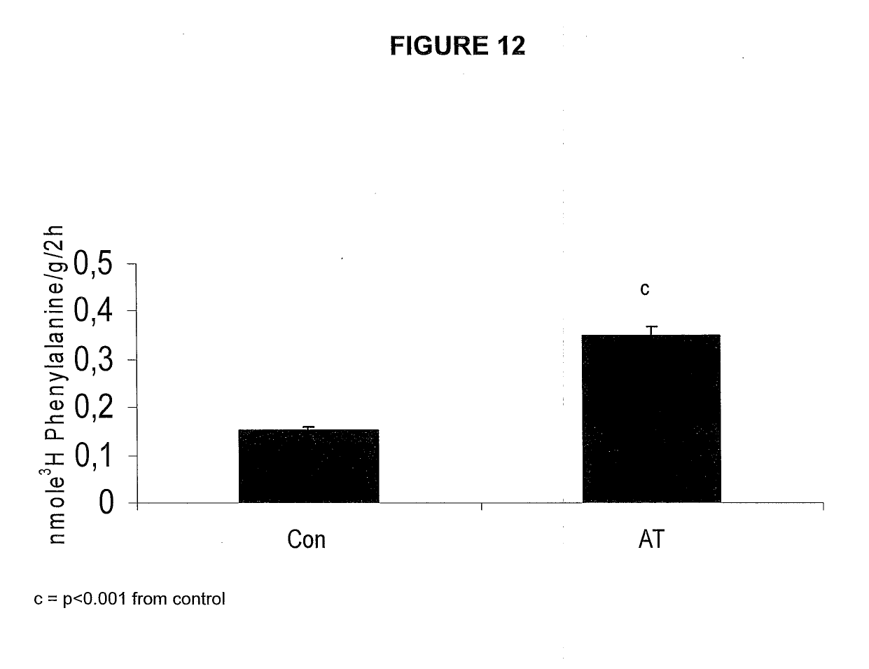

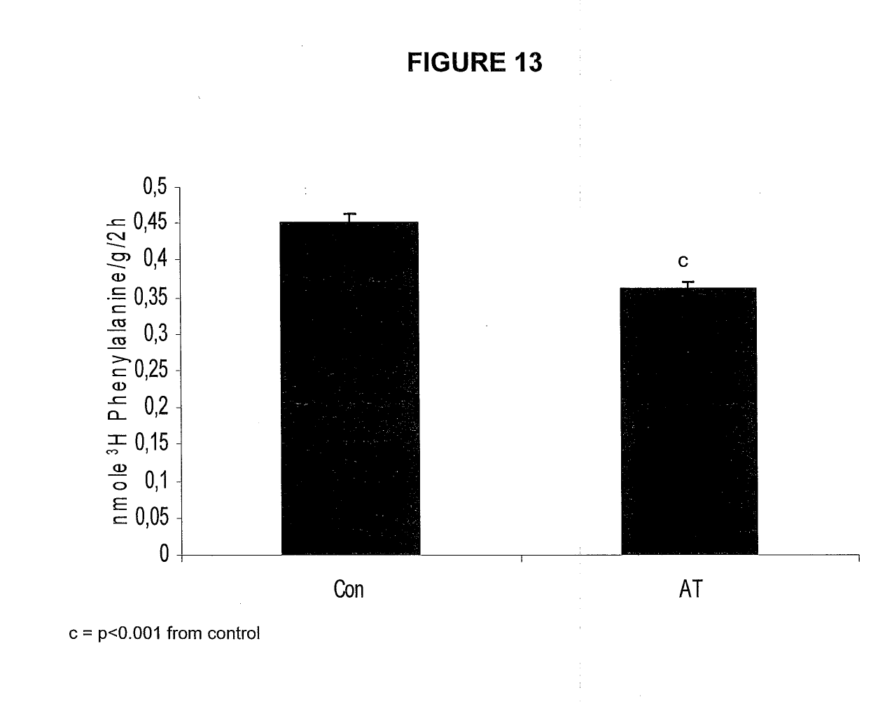

Results on protein synthesis and protein degradation. The results are shown in FIG. 10-13. As observed in Example 4, the weight loss was significantly lower in mice treated with alpha-trinositol compared to the control group (FIG. 10). The gastrocnemius muscle weight in MAC16 tumour-bearing animals was significantly higher compared to the control (FIG. 11). This was further verified by a significant increase (50%) (p<0.001) of protein synthesis in the gastrocnemius muscle in the mice treated with alpha-trinositol (FIG. 12) and a significant decrease (20%) (p<0.001) in protein degradation in the gastrocnemius muscle in the mice treated with alpha-trinositol compared (FIG. 13). These results suggest that alpha-trinositol increases lean body mass through an increase in protein synthesis and a decrease in protein degradation in the gastrocnemius muscle.

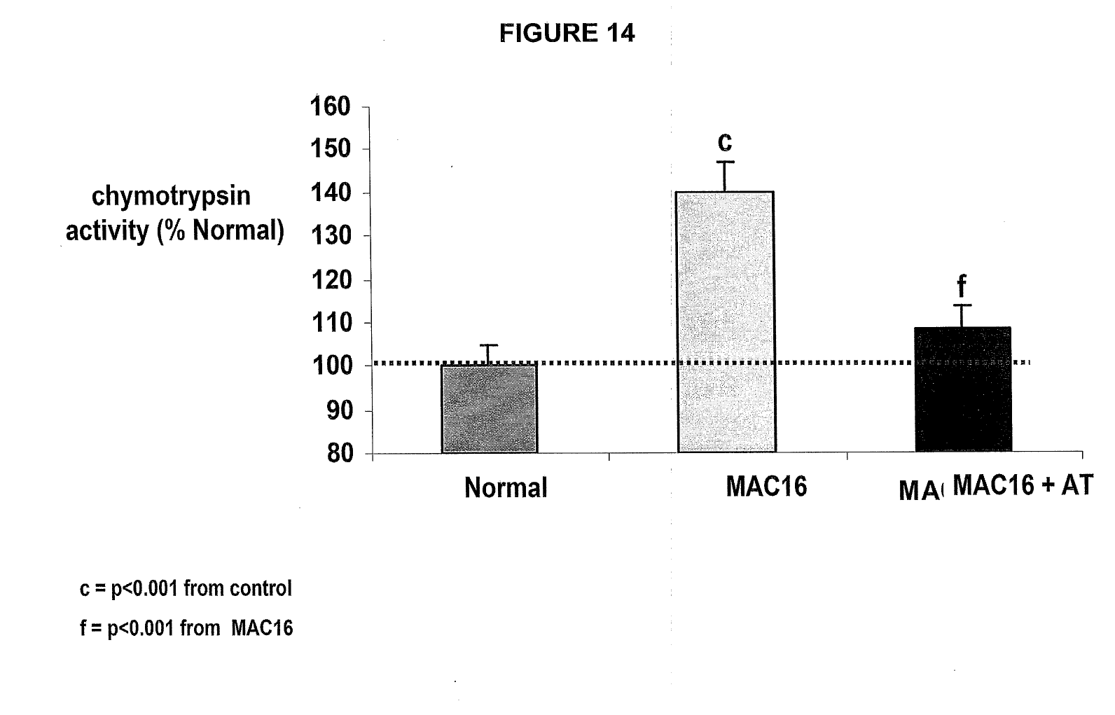

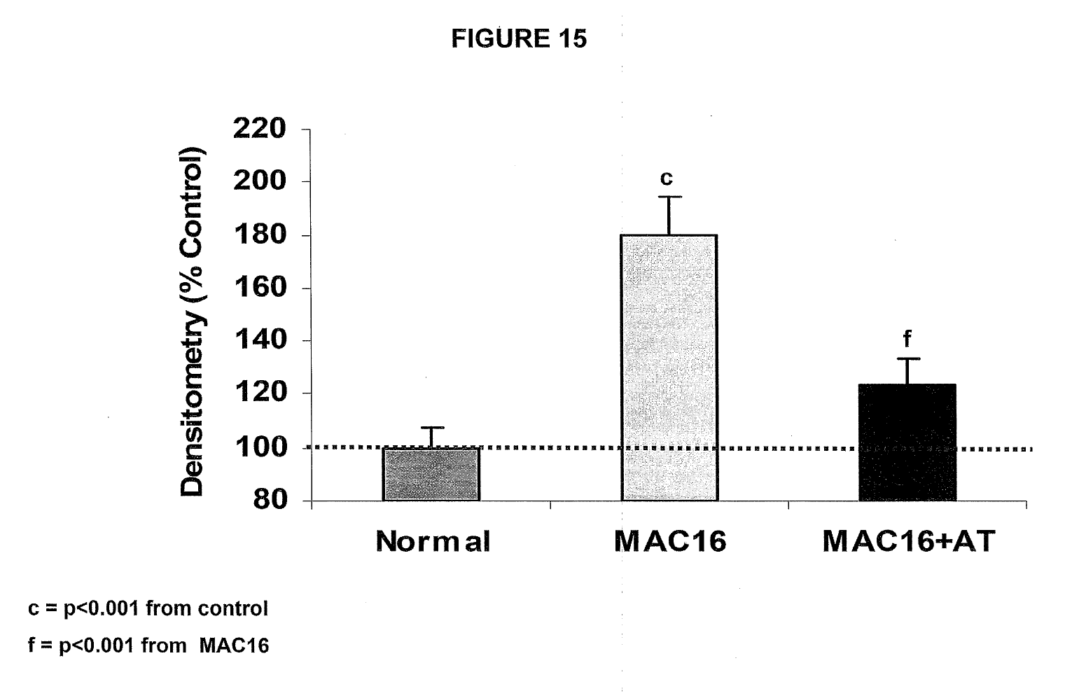

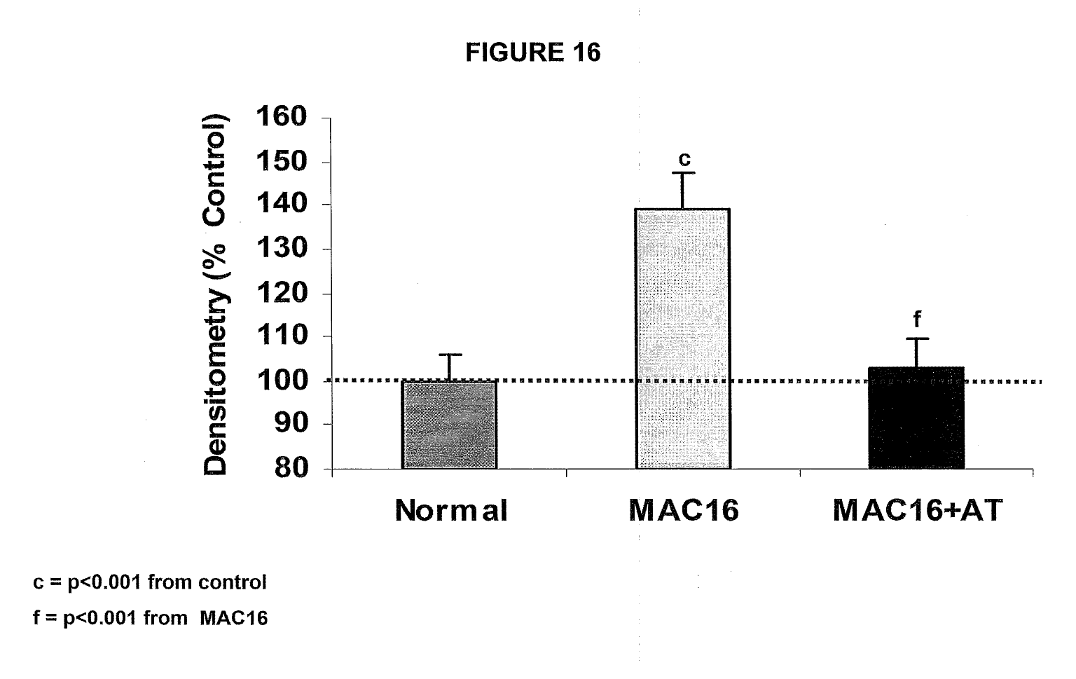

Results on 20S proteasome activity. In the gastrocnemius muscle there was a significant increase in the chymotrypsin activity (FIG. 14) as indicative of an increase in the 20S proteasome activity. After a 4 days treatment with alpha-trinositol the chymotrypsin activity in the tumour-bearing animal was reduced down to levels found in normal non-tumour bearing mice. This was further confirmed by the measurement of the expression of the 20S proteasome α-subunits (FIG. 15) and the expression of p42 (FIG. 16) and suggests that alpha-trinositol down-regulates the increased activity of the ubiquitin-proteasome pathway observed in cachectic animals.

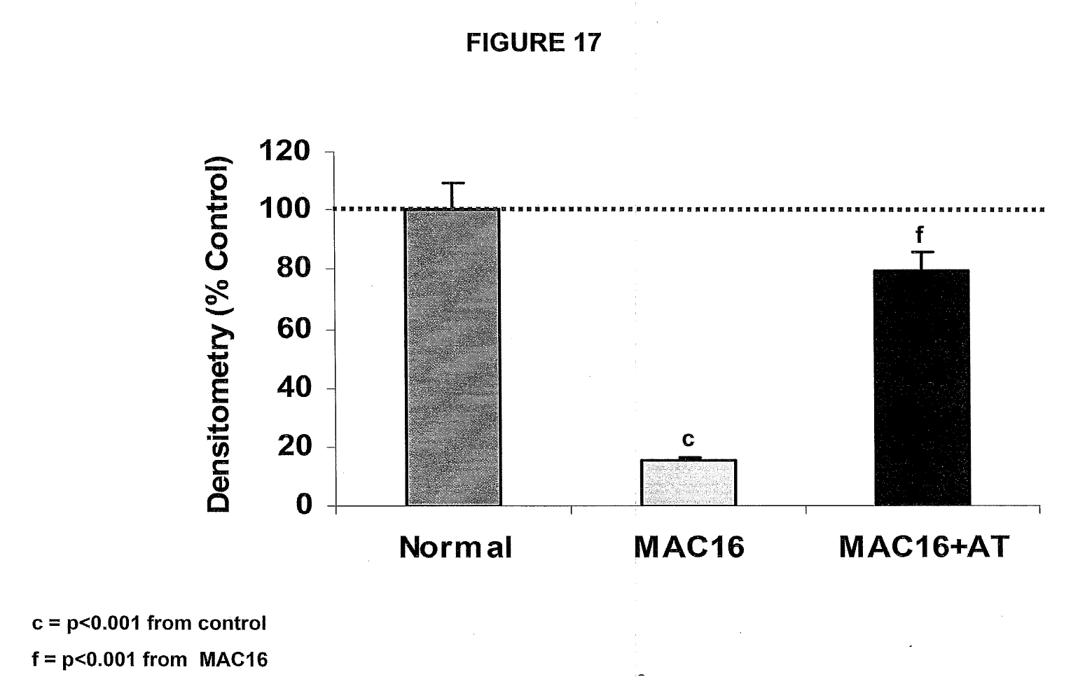

Results from the Western blot analysis. The measurement of myosin expression correlated inversely with the levels of proteasome components. The myosin expression was reduced by 90% (FIG. 17) in tumour-bearing mice and returned to values at the same level as in non-tumour animals after treatment with alpha-trinositol for 4 days.

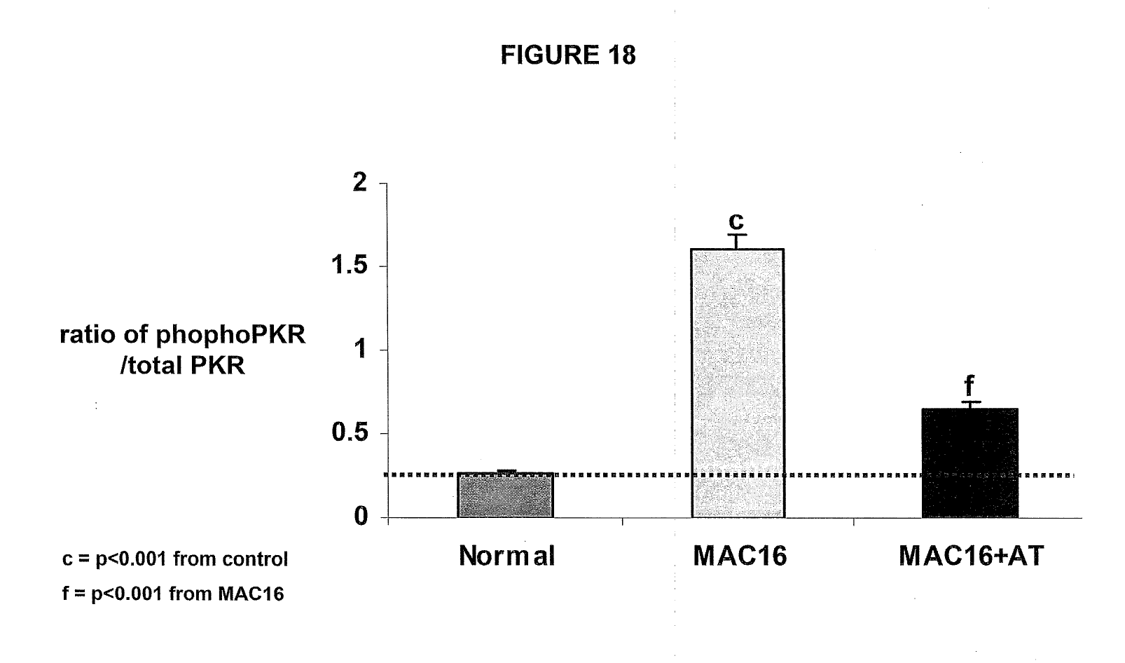

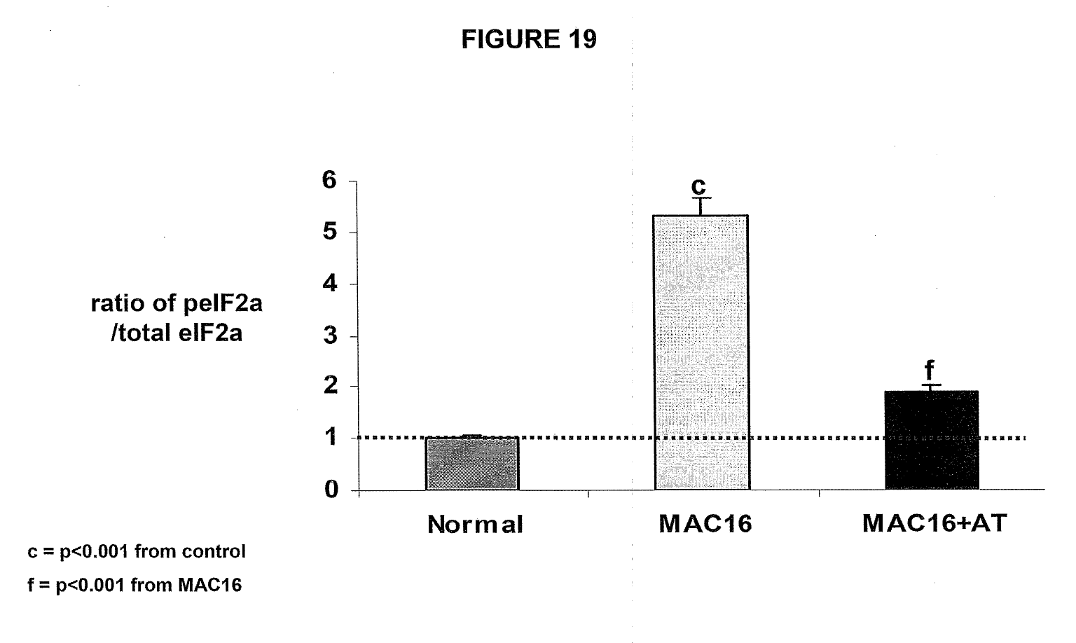

The expression of both phopho PKR (FIG. 18) and eIF2α (FIG. 19) correlated with the increase in protein synthesis and showed a significant decrease after treatment with alpha-trinositol in tumour-bearing animals.

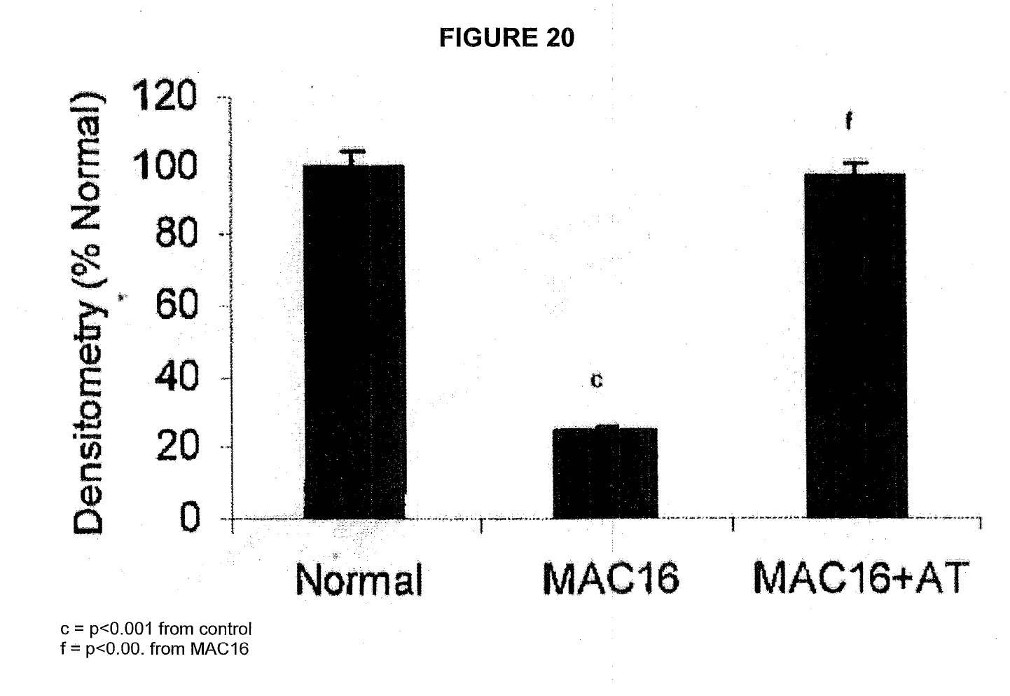

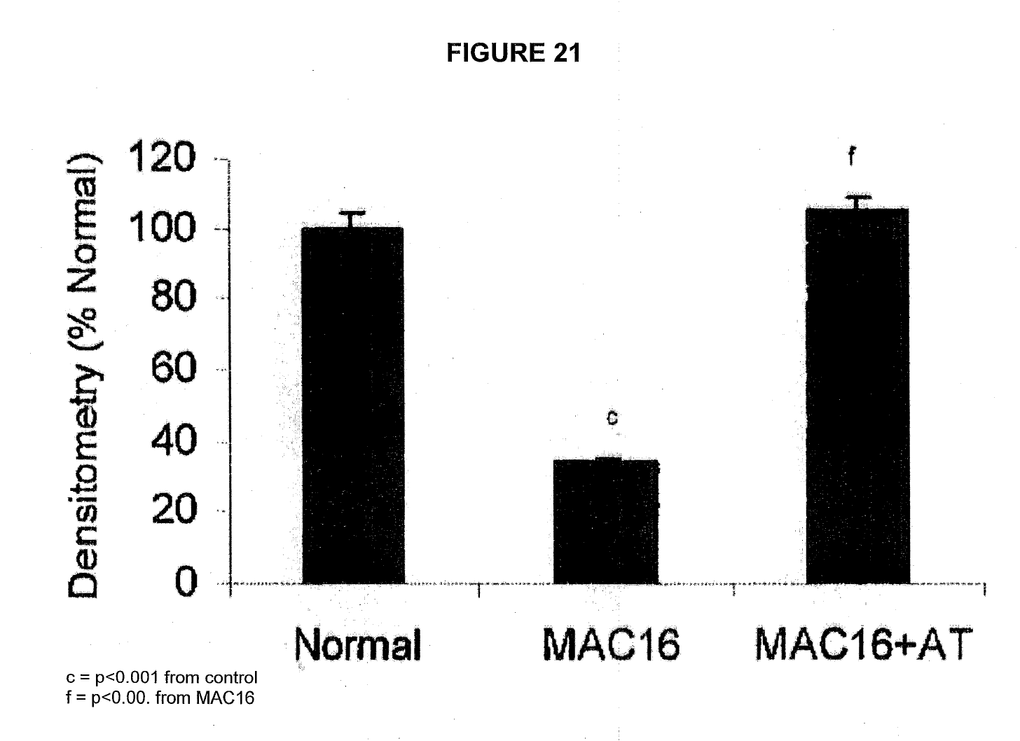

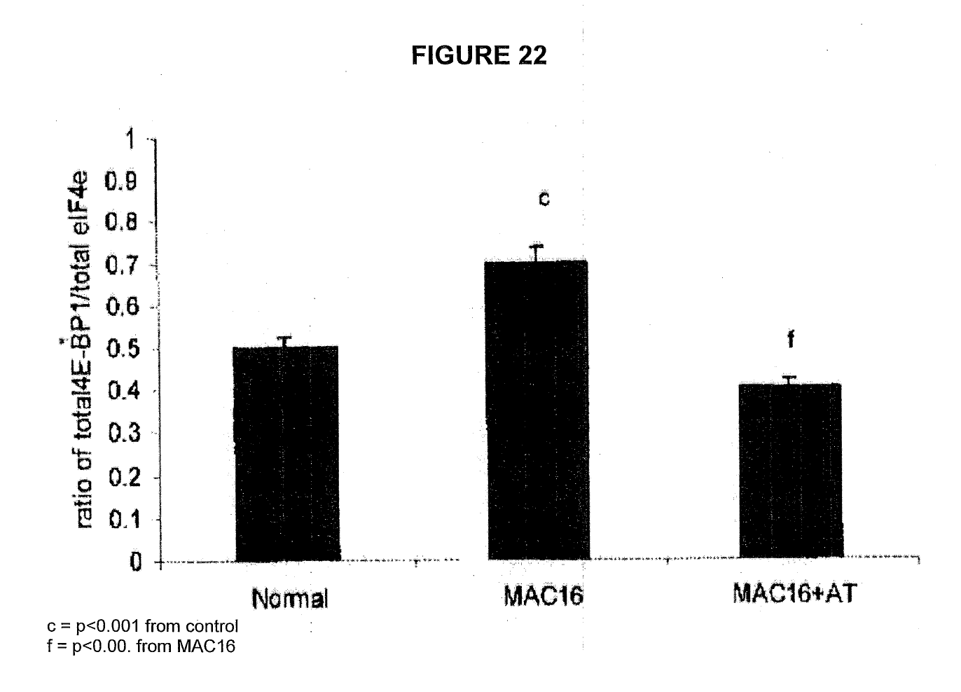

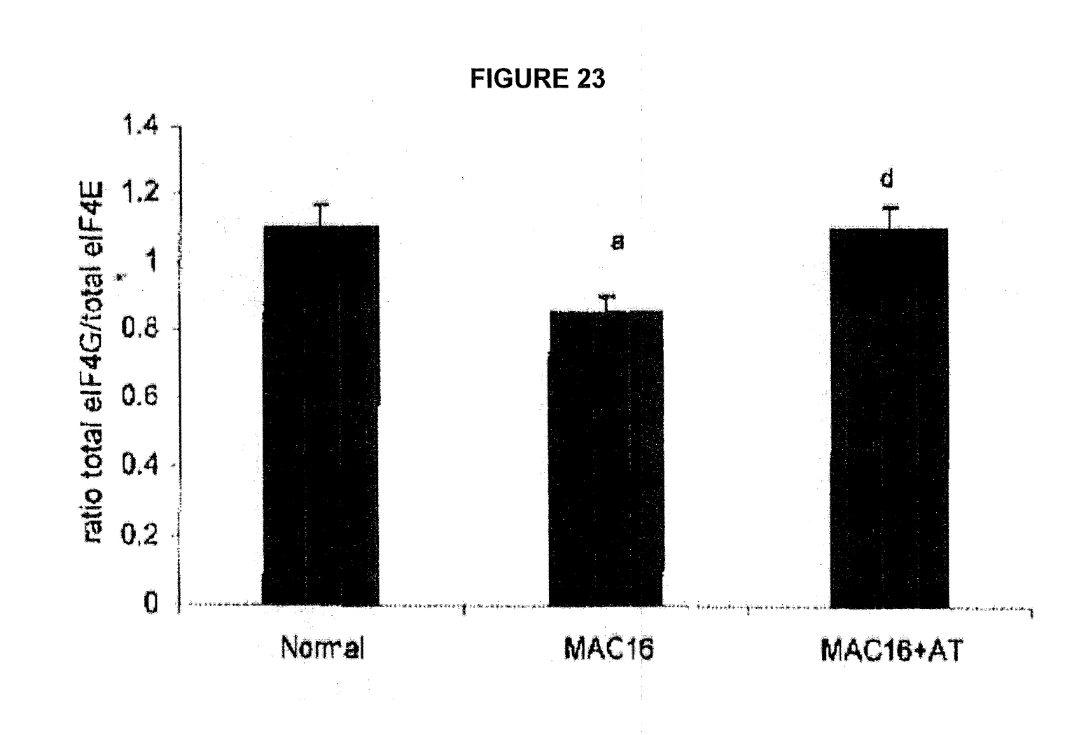

There was also a significant reduction in the level of the phosphorylated (Ser²⁴⁴⁸) form of mTOR in gastrocnemius muscle of mice bearing the MAC16 tumour (FIG. 20), which was completely reversed up to the values found in non tumour-bearing animals after 4 days treatment with alpha-trinositol. The effect of cachexia on the amount of eIF4E available for formation of the active eIF4G.eIF4E complex in gastrocnemius muscle and the effect of AT was also studied. Tumour-bearing animals showed a 60% reduction in the level of phosphorylation of 4E-BP1 (Thr^{37/46}) (FIG. 21), but there was no effect on phosphorylation of eIF4E (Ser²⁰⁹) (FIG. 22). Weight loss increased the amount of 4E-BP1 associated with eIF4E, and decreased formation of the active eIF4G.eIF4E complex (FIG. 23) in tumour-bearing animals. These effects were completely attenuated by the treatment with alpha-trinositol, such that the levels of eIF4F were the same as in non-tumour bearing controls (FIG. 22).

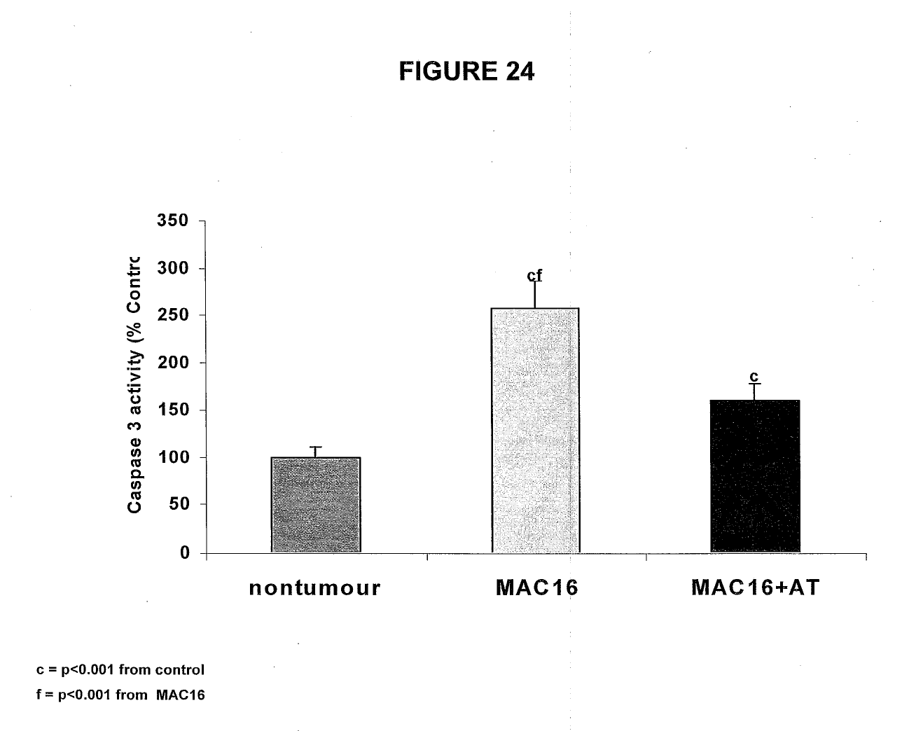

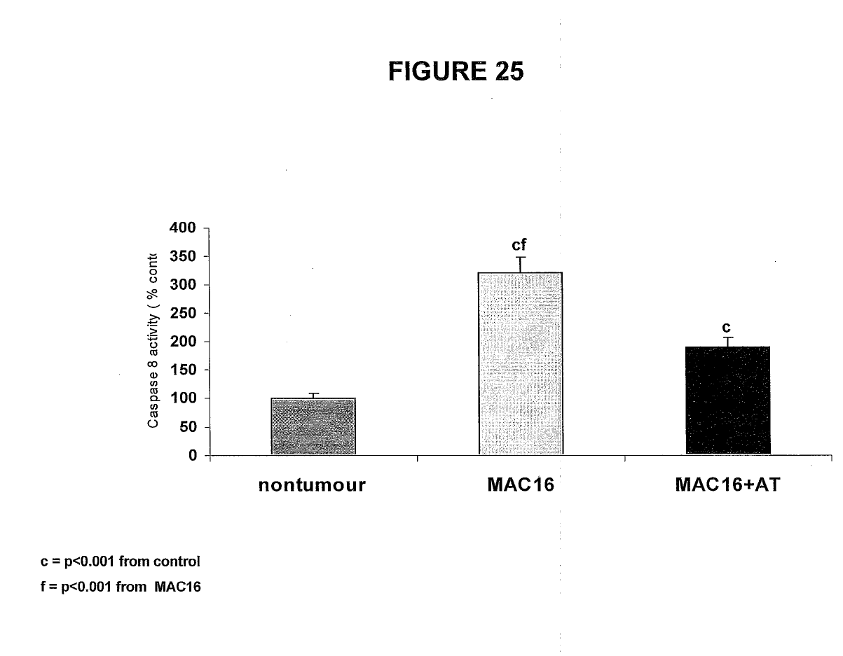

Results from the determination of caspase activity. The activity of caspase-3 (FIG. 24) and caspase-8 (FIG. 25) was elevated 2.5 to 3-fold in gastrocnemius muscle of mice bearing the MAC16 tumour, compared with non tumour-bearing animals. In tumour-bearing animals treated with alpha-trinositol this level was significantly reduced, although the levels were still significantly higher than those found in non-tumour-bearing animals.

### Example 5

### Treatment of Cachectic Mice with Lipid-Soluble Alpha-Trinositol (1-D-Tri-O-Hexanoyl-Myo-Inositol 1,2,6-Triphosphate)

Materials. Lipid soluble alpha trinositol (1-D-tri-O-hexanoyl-myo-inositol 1,2,6-triphosphate) was formed by further esterification of 1-D-Myo-inositol 1,2,6-triphosphate.

Animals. Pure strain male NMRI mice (average body weight 25 g) were transplanted with fragments of the MAC16 tumour subcutaneously into the flank by means of a trochar, selected from donor animals with established weight loss (Bibby M C et al., Characterization of transplantable adenocarcinoma in the mouse colon producing cachexia in the recipient animals. J Natl Cancer Inst 78 (1987) 539-546). Transplanted animals were given a rat and mouse breeding diet (Special Diet Services, Witham, UK) and water at lib. Weight loss was evident 10 to 12 days after tumour implantation. Just prior to the development of weight loss 24 animals were randomized into three groups of six animals each.

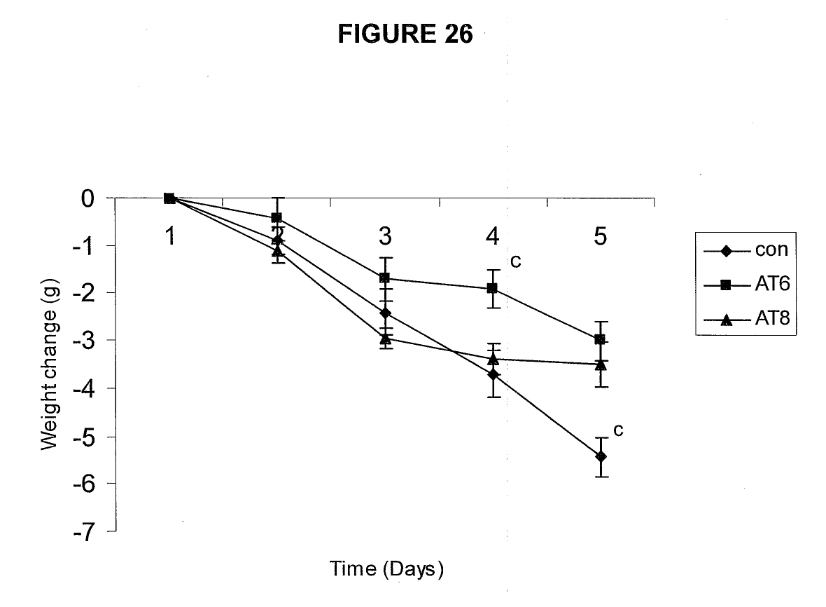

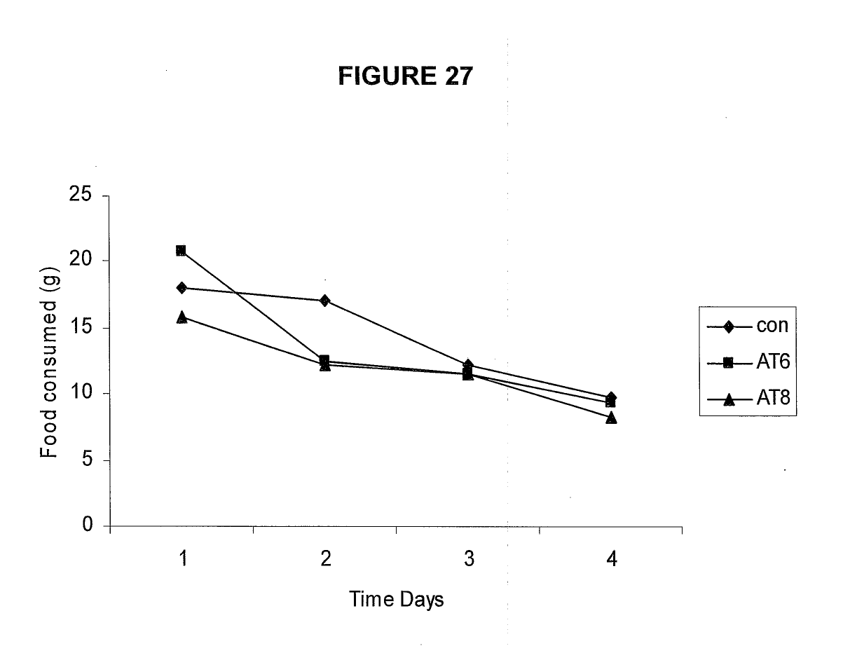

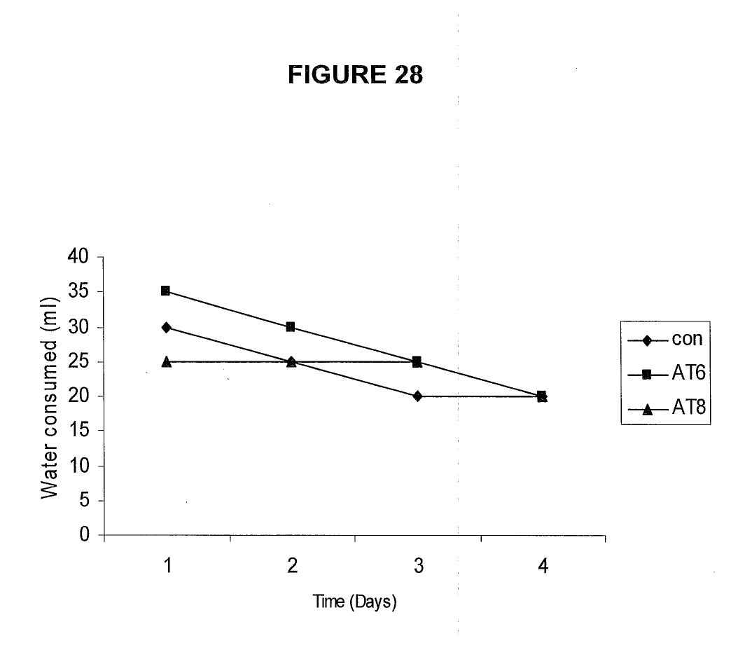

Administration of alpha-trinositol. A first experiment was started when the animals had lost about 5% of body weight. Aliquots of lipid soluble alpha-trinositol stock solution corresponding to doses of 6 mg/kg body weight and 8 mg/kg body weight for a 25 g animal were administered subcutaneously three times per day at 8.00, 12.00 and 16.00 hrs. The third group (control) was given an intravenous injection of 10 μl of PBS. Body weight (FIG. 26), tumour volume, and food (FIG. 27) and water (FIG. 28) intake were determined daily (for 5 days) after the last injection. Day 0 is the day of transplant and day 1 is the start of the experiment. The experiment was terminated on day 5 due to loss of controls.

Results. The results are shown in FIG. 26-28. FIG. 26 shows the weight change in MAC16 tumour-bearing mice treated with lipid soluble alpha-trinositol. The mice receiving lipid-soluble alpha-trinositol (both 8 mg/kg and 6 mg/kg) had significantly lower weight loss compared to the control group. In the group treated with the highest concentration of lipid soluble alpha trinositol this difference was significant already after 4 days. The average weight loss in the control group was about 6 g in 5 days and the corresponding weight loss in the group treated with the highest concentration of lipid soluble alpha trinositol was about 3 g.

The increase in tumour volume was similar between the two groups treated with lipid soluble alpha trinositol but slightly higher in the control group.

Thus, similarly to alpha trinositol, lipid soluble alpha trinositol is effective in attenuating weight loss in cachectic mice but have less effect on tumour growth rate. This confirms the results from the administration of water-soluble AT that the anti-cachexic effect is not caused by a tumour inhibitory effect, i.e. AT inhibits cachexia through a tumour independent pathway.

As can be seen in FIGS. 27 and 28, the food and water intake were not affected by the treatment.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

1.-62. (canceled)

63. A method of treatment of cachexia in a mammal, comprising the administration of a pharmacologically effective amount of a compound, said compound comprising a high density, negatively charged domain of vicinally oriented radicals.

64. The method according to claim 63, wherein the negatively charged domain comprises three or more vicinal phosphorus-containing radicals.

65. The method according to claim 63, wherein the phosphorus-containing radical has the general formula I 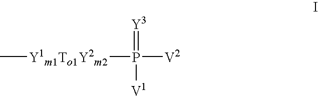
or the general formula II 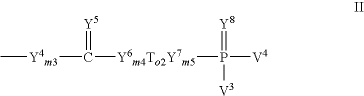
wherein
V¹ to V⁴ are Y⁹ₘ₆Tₒ₃U,
Tₒ₁ to Tₒ₃ are (CH₂)ₙ, CH═CH, or CH₂CH═CHCH₂,
o1 to o3 are 0 to 1,
n is 0 to 4,
U is R¹Y¹⁰ₘ₇, CY¹¹Y¹²R², SY¹³Y¹⁴Y¹⁵R³, PY¹⁶Y¹⁷Y¹⁸R⁴R⁵, Y¹⁹PY²⁰Y²¹Y²²R⁶R⁷, CH₂NO₂, NHSO₂R⁸ or NHCY²³Y²⁴R⁹,
m1 to m7 are 0 to 1,
Y¹ to Y²⁴ are NHR¹⁰, NOR¹¹, O or S,
and wherein R¹ to R¹¹ are
i) hydrogen;
ii) a straight or branched saturated or unsaturated alkyl residue of 1-22 carbon atoms;
iii) a saturated or unsaturated aromatic or non-aromatic homo- or heterocyclic residue of 3-22 carbon atoms and 0-5 hetero atoms selected from nitrogen, oxygen and sulfur;
iv) a straight or branched saturated or unsaturated alkyl residue of 1-22 carbon atoms comprising a saturated or unsaturated aromatic or non-aromatic homo- or heterocyclic substituent of 3-22 carbon atoms and 0-5 hetero atoms selected from nitrogen, oxygen and sulfur; or
v) an aromatic or non-aromatic homo- or heterocyclic residue of 3-22 carbon atoms and 0-5 heteroatoms selected from nitrogen, oxygen and sulfur, comprising a straight or branched saturated or unsaturated alkyl substituent of 1-22 carbon atoms.

66. The method according to claim 65, wherein one or several of the residues and/or substituents of R¹ to R¹¹, ii)-v), are substituted with 1 to 6 of hydroxy, alkoxy, aryloxy, acyloxy, carboxy, alkoxycarbonyl, alkoxycarbonyloxy, aryloxycarbonyl, aryloxycarbonyloxy, carbamoyl, fluoro, chloro, bromo, azido, cyano, oxo, oxa, amino, imino, alkylamino, arylamino, acylamino, arylazo, nitro, alkylthio and/or alkylsulfonyl.

67. The method according to claim 65, wherein one or several of the straight or branched saturated or unsaturated alkyl residues and substituents of R¹ to R¹¹, ii), iv), v) are selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methyl-heptyl, 2-methyloctyl, 2-methylnonyl, 2-methyldecyl, 2-methyl-eicosyl, 2-ethylbutyl, 2-ethylpentyl, 2-ethyl-hexyl, 2-ethylheptyl, 2-ethyloctyl, 2-ethylnonyl, 2-ethyldecyl, 2-ethyleicosyl, tertbutyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl, and doeicosynyl.

68. The method according to claim 65, wherein a saturated aromatic or non-aromatic homo- or heterocyclic of R¹ to R¹¹, iii)-v), is selected from the group consisting of cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl and a carbohydrate.

69. The method according to claim 64, wherein the phosphorus-containing radical has the general formula III 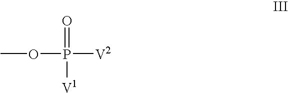
wherein V¹ and V² are, independent of each other, selected from the group consisting of OH, (CH₂)ₚOH, COOH, CONH₂, CONOH, (CH₂)ₚCOOH, (CH₂)ₚCONH₂, (CH₂)ₚCONOH, (CH₂)ₚSO₃H, (CH₂)ₚSO₃, NH₂, (CH₂)ₚNO₂, (CH₂)ₚPO₃H₂, O(CH₂)ₚOH, O(CH₂)ₚCOOH, O(CH₂)ₚCONH₂, O(CH₂)ₚCONOH, (CH₂)ₚSO₃H, O(CH₂)ₚSO₃NH₂, O(CH₂)ₚNO₂, O(CH₂)ₚPO₃H₂, and CF₂COOH, and p is 1 to 4.

70. The method according to claim 63, wherein the compound is a pharmaceutically acceptable phosphate, phosphonate or phosphinate.

71. The method according to claim 70, wherein the compound is a sodium, potassium, calcium or magnesium salt of the phosphate, phosphonate or phosphinate.

72. The method according to claim 69, wherein the domain of high density negatively charged vicinally oriented radicals is linked to a cyclic moiety comprising a saturated or unsaturated aromatic or non-aromatic homo- or heterocyclic ring.

73. The method according to claim 72, wherein heteroatom(s) of the heterocyclic ring of the cyclic moiety is/are, independently of each other, selected from the group consisting of oxygen, nitrogen, sulphur, and selenium.

74. The method according to claim 72, wherein the cyclic moiety comprises from 4 to 24 atoms.

75. The method according to claim 72, wherein the cyclic moiety is selected from the group consisting of cyclopentane, cyclohexane, cycloheptane, cyclooctane, inositol, monosascharide, disaccharide, trisaccharide, tetrasaccharide, arabinitol, piperidine, tetra-hydrothiopyran, 5-oxotetrahydrothiopyran, 5,5-dioxotetrahydro-thiopyran, tetrahydroselenopyran, tetrahydrofuran, pyrrolidine, tetrahydrothiophene, 5-oxotetrahydrothiophene, 5,5-dioxotetrahydrothiophene, tetrahydroselenophene, benzene, cumene, mesitylene, naphtalene and phenantrene.

76. The method according to claim 75, wherein the cyclic moiety comprises one or more hydroxyl groups not bound to phosphorous-containing radicals of which at least one is derivatized in form of an ether or ester.

77. The method according to claim 75, wherein the cyclic moiety is selected from the group consisting of inositol, monosaccharide, disaccharide, trisaccharide, and tetrasaccharide.

78. The method according to claim 77, wherein the cyclic moiety is an inositol selected from the group consisting of allo-inositol, cis-inositol, epi-inositol, D/L-chiro-inositol, scylloinositol, myoinositol, mucoinositol and neoinositol.

79. The method according to claim 77, wherein the cyclic moiety is inositol and the compound is selected from the group consisting of a phosphate, a phosphonate or a phosphinate of inositol.

80. The method according to claim 79, wherein the number of phosphate, phosphonate or phosphinate radicals per inositol moiety is three or more.

81. The method according to claim 77, wherein the cyclic moiety is inositol and the inositol is selected from the group consisting of inositol-trisphosphate, inositol-tris(carboxymethyl-phosphate), inositol-tris(carbomethylphosphonate), inositol-tris(hydroxymethylphosphonate), tri-O-methyl-inositol-trisphosphate, tri-O-hexyl-inositol-trisphosphate, tri-O-butyl-inositol-trisphosphate, tri-O-pentyl-inositol-trisphosphate, tri-O-isobutyl-inositol-trisphosphate, tri-O-propyl-inositol-trisphosphate, tri-O-(6-hydroxy-4-oxa)hexyl-inositol-trisphosphate, tri-O-3-(ethylsulfonyl)propyl-inositol-trisphosphate, tri-O-3-hydroxypropyl-inositol-trisphosphate, tri-O-(6-hydroxy)-hexyl-inositol-trisphosphate, tri-O-phenylcarbamoyl-inositol-trisphosphate, tri-O-propyl-inositol-tris(carboxymethylphosphate), tri-O-butyl-inositol-tris(carboxymethylphosphate), tri-O-isobutyl-inositol-tris(carboxymethyl-phosphate), tri-O-pentyl-inositol-tris(carboxymethylphosphate), tri-O-hexyl-inositol-tris(carboxymethylphosphate), tri-O-propyl-inositol-tris(carboxymethylphosphonate), tri-O-butyl-inositol-tris(carboxymethyl-phosphonate), tri-O-isobutyl-inositol-tris(carboxymethylphosphonate), tri-O-pentyl-inositol-tris(carboxymethylphosphonate), tri-O-hexyl-inositol-tris(carboxymethylphosphonate), tri-O-propyl-inositol-tris(hydroxymethyl-phosphonate), tri-O-butyl-inositol-tris(hydroxymethylphosphonate), tri-O-isobutyl-inositol-tris(hydroxymethylphosphonate), tri-O-pentyl-inositol-tris(hydroxymethylphosphonate), and tri-O-hexyl-myo-inositol-tris(hydroxymethyl-phosphonate).

82. The method according to claim 78, wherein the cyclic moiety is a myoinositol selected from the group consisting of D-myo-inositol-1,2,6-trisphosphate, D-myo-inositol-1,2,6-tris(carboxymethyl-phosphate), D-myo-inositol-1,2,6-tris(carbomethylphosphonate), D-myo-inositol-1,2,6-tris(hydroxymethylphosphonate), D-3,4,5-tri-O-methyl-myo-inositol-1,2,6-trisphosphate, D-3,4,5-tri-O-hexyl-myo-inositol-1,2,6-trisphosphate, D-3,4,5-tri-O-butyl-myo-inositol-1,2,6-trisphosphate, D-3,4,5-tri-O-pentyl-myo-inositol-1,2,6-trisphosphate, D-3,4,5-tri-O-isobutyl-myo-inositol-1,2,6-trisphosphate, D-3,4,5-tri-O-propyl-myo-inositol-1,2,6-trisphosphate, D-3,4,5-tri-O-(6-hydroxy-4-oxa)hexyl-myo-inositol-1,2,6-trisphosphate, D-3,4,5-tri-O-3-(ethylsulfonyl)propyl-myo-inositol-1,2,6-trisphosphate, D-3,4,5-tri-O-3-hydroxypropyl-myo-inositol-1,2,6-trisphosphate, D-3,4,5-tri-O-(6-hydroxy)-hexyl-myo-inositol-1,2,6-trisphosphate, D-5-O-hexyl-myo-inositol-1,2,6-trisphosphate, D-3,4,5-tri-O-phenylcarbamoyl-myo-inositol-1,2,6-trisphosphate, D-3,4,5-tri-O-propyl-myo-inositol-1,2,6-tris(carboxymethylphosphate), D-3,4,5-tri-O-butyl-myo-inositol-1,2,6-tris(carboxymethylphosphate), D-3,4,5-tri-O-isobutyl-myo-inositol-1,2,6-tris(carboxymethyl-phosphate), D-3,4,5-tri-O-pentyl-myo-inositol-1,2,6-tris(carboxymethylphosphate), D-3,4,5-tri-O-hexyl-myo-inositol-1,2,6-tris(carboxymethylphosphate), D-3,4,5-tri-O-propyl-myo-inositol-1,2,6-tris(carboxymethylphosphonate), D-3,4,5-tri-O-butyl-myo-inositol-1,2,6-tris(carboxymethyl-phosphonate), D-3,4,5-tri-O-isobutyl-myo-inositol-1,2,6-tris(carboxymethylphosphonate), D-3,4,5-tri-O-pentyl-myo-inositol-1,2,6-tris(carboxymethylphosphonate), D-3,4,5-tri-O-hexyl-myo-inositol-1,2,6-tris(carboxymethylphosphonate), D-3,4,5-tri-O-propyl-myo-inositol-1,2,6-tris(hydroxymethyl-phosphonate), D-3,4,5-tri-O-butyl-myo-inositol-1,2,6-tris(hydroxymethylphosphonate), D-3,4,5-tri-O-isobutyl-myo-inositol-1,2,6-tris(hydroxymethylphosphonate), D,-3,4,5-tri-O-pentyl-myo-inositol-1,2,6-tris(hydroxymethylphosphonate), D-3,4,5-tri-O-hexyl-myo-inositol-1,2,6-tris(hydroxymethyl-phosphonate), D-3,4,5-tri-O-propanoyl-myo-inositol-1,2,6-tris(carboxymethylphosphate), D-3,4,5-tri-O-butanoyl-myo-inositol-1,2,6-tris(carboxymethylphosphate), D-3,4,5-tri-O-isobutanoyl-myo-inositol-1,2,6-tris(carboxymethyl-phosphate), D-3,4,5-tri-O-pentanoyl-myo-inositol-1,2,6-tris(carboxymethylphosphate), D-3,4,5-tri-O-hexanoyl-myo-inositol-1,2,6-tris(carboxymethylphosphate), D-3,4,5-tri-O-propanoyl-myo-inositol-1,2,6-tris(carboxymethylphosphonate), D-3,4,5-tri-O-butanoyl-myo-inositol-1,2,6-tris(carboxymethyl-phosphonate), D-3,4,5-tri-O-isobutanoyl-myo-inositol-1,2,6-tris(carboxymethylphosphonate), D-3,4,5-tri-O-pentanoyl-myo-inositol-1,2,6-tris(carboxymethylphosphonate), D-3,4,5-tri-O-hexanoyl-myo-inositol-1,2,6-tris(carboxymethylphosphonate), D-3,4,5-tri-O-propanoyl-myo-inositol-1,2,6-tris(hydroxymethyl-phosphonate), D-3,4,5-tri-O-butanoyl-myo-inositol-1,2,6-tris(hydroxymethylphosphonate), D-3,4,5-tri-O-isobutanoyl-myo-inositol-1,2,6-tris(hydroxymethylphosphonate), D,-3,4,5-tri-O-pentanoyl-myo-inositol-1,2,6-tris(hydroxymethylphosphonate), and D-3,4,5-tri-O-hexanoyl-myo-inositol-1,2,6-tris(hydroxymethyl-phosphonate).

83. The method according to claim 82, wherein the myoinsoitol is selected from the group consisting of myo-inositol-1,2,6-trisphosphate and myo-inositol-1,2,3-trisphosphate.

84. The method according to claim 76, wherein at least one of the hydroxyl groups is derivatized to form an ester having the general formula IV

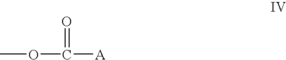

85. The method according to claim 84, wherein A is selected from the group consisting of:
i) a straight or branched saturated or unsaturated alkyl residue containing 1 to 24 carbon atoms selected from the group consisting of methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methylheptyl, 2-methyloctyl, 2-methylnonyl, 2-methyldecyl, 2-methyleicosyl, 2-ethylbutyl, 2-ethylpentyl, 2-ethylhexyl, 2-ethylheptyl, 2-ethyloctyl, 2-ethylnonyl, 2-ethyldecyl, 2-ethyleicosyl, tert-butyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl and doeicosynyl;
ii) a saturated or unsaturated aromatic or non-aromatic homo- or heterocyclic residue or substituent selected from the group consisting of cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl and carbohydrate;
iii) (CH₂)ₙOR¹² where n is an integer between 1 and 10 and where R¹² is hydrogen, a substituted or unsubstituted straight or branched alkyl, cycloalkyl, aryl or alkaryl;
iv) (CH₂)ₙZ(CH₂)ₘOR¹² where n and m is an integer between 1 and 10, where Z is oxygen or sulphur and where R¹² is hydrogen, a substituted or unsubstituted straight or branched alkyl, cycloalkyl, aryl or alkaryl;
v) (CH₂)ₙOCOR¹² where n is an integer between 1 and 10 and where R¹² is hydrogen, a substituted or unsubstituted straight or branched alkyl, cycloalkyl, aryl or alkaryl;
vi) (CH₂)ₙCOOR¹² where n is an integer between 1 and 10 and where R¹² is hydrogen, a substituted or unsubstituted straight or branched alkyl, cycloalkyl, aryl or alkaryl;
vii) (CH₂)ₙOCOOR¹² where n is an integer between 1 and 10 and where R¹² is hydrogen, a substituted or unsubstituted straight or branched alkyl, cycloalkyl, aryl or alkaryl;
viii) (CH₂)ₙR¹² where n is an integer between 1 and 10 and where R¹² is hydrogen, a substituted or unsubstituted straight or branched alkyl, cycloalkyl, aryl or alkaryl; and
ix) (CH₂)ₙOCONR¹²R¹³ where n is an integer between 1 and 10 and where R¹² and R¹³ are hydrogen, a substituted or unsubstituted straight or branched alkyl, cycloalkyl, aryl or alkaryl.

86. The method according to claim 75, wherein the compound is selected from the group consisting of a phosphate, a phosphonate or a phosphinate of cyclohexane.

87. The method according to claim 75, wherein the compound is an inositol triphosphate selected from the group consisting of tri-O-hexanoyl-inositol-trisphosphate, tri-O-butanoyl-inositol-trisphosphate, tri-O-pentanoyl-inositol-trisphosphate, tri-O-(4-hydroxy)pentanoyl-inositol-trisphosphate, tri-O-isobutanoyl-inositol-trisphosphate, tri-O-propanoyl-inositol-trisphosphate, tri-O-(6-hydroxy-4-oxa)hexanoyl-inositol-trisphosphate, tri-O-3-(ethylsulfonyl)propanoyl-inositol-trisphosphate, tri-O-3-hydroxypropanoyl-inositol-trisphosphate, tri-O-(6-hydroxy)-hexanoyl-inositol-trisphosphate, tri-O-phenylcarbamoyl-inositol-trisphosphate, tri-O-dodecanoyl-inositol-trisphosphate, tri-O-(2-acetoxy)benzoylcarbamoyl-inositol-trisphosphate, tri-O-butylcarbamoyl-inositol-trisphosphate, tri-O-methylcarbamoyl-inositol-trisphosphate, and tri-O-phenylcarbamoyl-inositol-trisphosphate.

88. The method according to claim 75, wherein the compound is selected from the group consisting of D-3,4,5-tri-O-hexanoyl-myo-inositol-1,2,6-trisphosphate, D-3,4,5-tri-O-butanoyl-myo-inositol-1,2,6-trisphosphate, D-3,4,5-tri-O-pentanoyl-myo-inositol-1,2,6-trisphosphate, D-3,4,5-tri-O-(4-hydroxy)pentanoyl-myo-inositol-1,2,6-trisphosphate, D-3,4,5-tri-O-isobutanoyl-myo-inositol-1,2,6-trisphosphate, D-3,4,5-tri-O-propanoyl-myo-inositol-1,2,6-trisphosphate, D-3,4,5-tri-O-(6-hydroxy-4-oxa)hexanoyl-myo-inositol-1,2,6-trisphosphate, D-3,4,5-tri-O-3-(ethylsulfonyl)propanoyl-myo-inositol-1,2,6-trisphosphate, D-3,4,5-tri-O-3-hydroxypropanoyl-myo-inositol-1,2,6-trisphosphate, D-3,4,5-tri-O-(6-hydroxy)-hexanoyl-myo-inositol-1,2,6-trisphosphate, D-3,4,5-tri-O-phenylcarbamoyl-myo-inositol-1,2,6-trisphosphate, D-3,4,5-tri-O-dodecanoyl-myo-inositol-1,2,6-trisphosphate, D-3,4,5-tri-O-(2-acetoxy)benzoylcarbamoyl-myo-inositol-1,2,6-trisphosphate, D-3,4,5-tri-O-butylcarbamoyl-myo-inositol-1,2,6-trisphosphate, D-3,4,5-tri-O-methylcarbamoyl-myo-inositol-1,2,6-trisphosphate, and D-3,4,5-tri-O-phenylcarbamoyl-myo-inositol-1,2,6-trisphosphate.

89. The method according to claim 88, wherein the compound is in the form of its sodium salt.

90. The method according to 77, wherein the cyclic moiety is a mono- or disaccharide selected from the group consisting of D/L-ribose, D/L-arabinose, D/L-xylose, D/L-lyxose, D/L-allose, D/L-altrose, D/L-glucose, D/L-mannose, D/L-gulose, D/L-idose, D/L-galactose, D/L-talose, D/L-ribulose, D/L-xylulose, D/L-psicose, D/L-sorbose, D/L-tagatose and D/L-fructose or a derivative thereof.

91. The method according to claim 90, wherein the compound is selected from the group consisting of a phosphate, a phosphonate or a phosphinate of a mono- or disaccharide.

92. The method according to claim 91, wherein the number of phosphate, phosphonate or phosphinate radicals per saccharide moiety is three or more.

93. The method according to claim 91, wherein the compound is selected from the group consisting of mannose-2,3,4-trisphosphate, galactose-2,3,4-trisphosphate, fructose-2,3,4-trisphosphate, altrose-2,3,4-trisphosphate and rhamnose-2,3,4-trisphosphate.

94. The method according to claim 72, wherein the compound is selected from the group consisting of R¹-6-O—R²-α-D-manno-pyranoside-2,3,4-trisphosphate, R¹-6-O—R²-α-D-galacto-pyranoside-2,3,4-trisphosphate, R¹-6-O—R²-α-D-altropyranoside-2,3,4-trisphosphate and R¹-6-O—R²-β-D-fructopyranoside-2,3,4-trisphosphate, wherein R¹ and R² independent of each other are selected from the group consisting of methyl, ethyl, propyl, butyl, pentyl and hexyl.

95. The method according to claim 94, wherein the compound is selected from the group consisting of methyl-6-O-butyl-α-D-mannopyranoside-2,3,4-trisphosphate, methyl-6-O-butyl-α-D-galactopyranoside-2,3,4-trisphosphate, methyl-6-O-butyl-α-D-glycopyranoside-2,3,4-trisphosphate, methyl-6-O-butyl-α-D-altropyranoside-2,3,4-trisphosphate, methyl-6-O-butyl-β-D-fructopyranoside-2,3,4-trisphosphate, 1,5-anhydro-D-arabinitol-2,3,4-trisphosphate, 1,5-anhydroxylitol-2,3,4-trisphosphate, 1,2-O-ethylene-β-D-fructopyranoside-2,3,4-trisphosphate, methyl-α-D-rhamno-pyranoside-2,3,4-trisphosphate, methyl-α-D-mannopyranoside-2,3,4-trisphosphate, methyl-6-O-butyl-α-D-mannopyranoside-2,3,4-tris-(carboxymethylphosphate), methyl-6-O-butyl-α-D mannopyranoside-2,3,4-tris(carboxymethylphosphonate), methyl-6-O-butyl-α-D-mannopyranoside-2,3,4-tris(hydroxymethyl-phosphonate), methyl-6-O-butyl-α-D-galactopyranoside-2,3,4-tris(carboxymethylphosphate), methyl-6-O-butyl-α-D-galacto-pyranoside-2,3,4-tris(carboxymethylphosphonate), methyl-6-O-butyl-α-D-galactopyranoside-2,3,4-tris(hydroxymethyl-phosphonate), methyl-6-O-butyl-α-D-glucopyranoside-2,3,4-tris(carboxymethylphosphate), methyl-6-O-butyl-α-D-glucopyranoside-2,3,4-tris(carboxymethylphosphonate), methyl-6-O-butyl-α-D-glucopyranoside-2,3,4-tris(hydroxy-methylphosphonate), methyl-6-O-butyl-α-D-altropyranoside-2,3,4-tris-(carboxymethylphosphate), methyl-6-O-butyl-α-D-altropyranoside-2,3,4-tris-(carboxymethylphosphonate), methyl-6-O-butyl-α-D-altropyranoside-2,3,4-tris-(hydroxyl-methylphosphonate), methyl-6-O-butyl-β-D-fructopyranoside-2,3,4-tris-(carboxymethylphosphate), methyl-6-O-butyl-β-D-fructopyranoside-2,3,4-tris-(carboxymethylphosphonate) and methyl-6-O-butyl-β-D-fructo-pyranoside-2,3,4-tris-(hydroxymethylphosphonate).

96. The method according to claim 75, wherein the compound is a phosphate, phosphonate or phosphinate comprising a heterocyclic moiety that is an arabinitol selected from the group consisting of 1,5-dideoxy-1,5-iminoarabinitol-2,3,4-trisphosphate, 1,5-dideoxy-1,5-iminoarabinitol-2,3,4-tris-(carboxymethylphosphate), 1,5-dideoxy-1,5-imino-arabinitol-2,3,4-tris(carboxymethyl-phosphonate), 1,5-dideoxy-1,5-iminoarabinitol-2,3,4-tris(hydroxymethylphosphonate), 1,5-dideoxy-1,5-imino-N-(2-phenylethyl)arabinitol-2,3,4-trisphosphate, 1,5-dideoxy-1,5-imino-N-(2-phenylethyl)-arabinitol-2,3,4-tris(carboxy-methylphosphate), 1,5-dideoxy-1,5-imino-N-(2-phenylethyl)-arabinitol-2,3,4-tris-(carboxy-methylphosphonate), and 1,5-dideoxy-1,5-imino-N-(2-phenyl-ethyl)arabinitol-2,3,4-tris(hydroxymethylphosphonate).

97. The method according to claim 63, wherein the cachexia is associated with cancer.

98. A pharmaceutical composition comprising a dose of the penta sodium salt of 1,2,6-D-myo inositol trisphosphate that is pharmacologically efficient in the treatment or prevention of cachexia, and a pharmaceutically acceptable fluid carrier.

99. A pharmaceutical composition comprising a dose of 1,2,6-D-myo inositol trisphosphate that is pharmacologically efficient in the treatment or prevention of cachexia, in a closed container and in crystalline or amorphous form.

100. A pharmaceutical composition comprising (i) a compound comprising a high density, negatively charged domain of vicinally oriented radicals, (ii) an analgesic agent, and (iii) a pharmaceutically acceptable carrier.

101. A composition according to claim 100, wherein the analgesic agent is an opoid agonist.

102. A nutritional composition for preventing cachexia or for preventing catabolic conditions associated with severe trauma, comprising:
i) an inositol triphosphate or an ester thereof, or a mono- or disaccharide having three or more phosphate radicals per saccharide moiety or an ester thereof; and
ii) at least one nutrient selected from the group consisting of lipid emulsions, fluid sources of amino acids, and carbohydrates.
